(19) United States
(12) Patent Application Publication
(10) Pub. No.: US 2015/0086509 A1
(43) Pub. Date: Mar. 26, 2015

(54) COMPOSITIONS AND METHODS FOR TREATING CANCER USING INTERFERON AND MAPK PATHWAY INHIBITOR

(71) Applicants: The Trustees of Columbia University in the City of New York, New York, NY (US); Sloan-Kettering Institute for Cancer Research, New York, NY (US)

(72) Inventors: Oren Litvin, Philadelphia, PA (US); Neal Rosen, New York, NY (US); Dana Pe'er, New York, NY (US)

(21) Appl. No.: 14/496,723

(22) Filed: Sep. 25, 2014

Related U.S. Application Data
(60) Provisional application No. 61/882,450, filed on Sep. 25, 2013.

(51) Int. Cl. A61K 38/21; G01N 33/574; C12Q 1/68; A61K 31/166

(52) U.S. Cl. CPC A61K 38/215; A61K 38/212; A61K 31/166; G01N 33/57496; C12Q 1/6886; C12Q 2600/158; C12Q 2600/106; G01N 2800/52; G01N 2333/912
USPC 424/85.6; 424/85.7; 435/7.1; 435/7.92; 435/7.4; 506/9; 435/6.11; 506/2

(57) ABSTRACT

The present invention provides for methods and compositions for treating cancer. A subject having cancer is administered an interferon and an inhibitor of mitogen-activated protein kinase (MAPK) signaling pathway. The combination of the interferon and the inhibitor of the MAPK pathway produces a synergistic effect on the cancer compared to the effect of the interferon or the inhibitor of the MAPK pathway alone. The activity of the interferon pathway, interferon expression levels and/or interferon locus copy number can be used as biomarkers for treatment of cancer by MAPK pathway inhibitors.

### CROSS REFERENCE TO RELATED APPLICATION

This application claims priority to U.S. Provisional Application No. 61/882,450 filed on Sep. 25, 2013, which is incorporated herein by reference in its entirety.

### GOVERNMENT LICENSE RIGHTS

This invention was made with government support under Grant No. R01CA164729 awarded by the National Institutes of Health and Grant No. 1U54CA121852-01A1 awarded by the National Centers for Biomedical Computing. The government has certain rights in the invention.

### FIELD OF THE INVENTION

The present invention relates to methods and compositions for the treatment of cancer and other conditions. In particular, the present invention relates to the combined use of an interferon and an inhibitor of the mitogen-activated protein kinase (MAPK) signaling pathway in treating cancer.

### BACKGROUND OF THE INVENTION

Advances in the identification and understanding of oncogenic pathways, as well as the development of highly specific drugs, allow clinicians to tailor treatments based on tumor genomics. However, drug response is variable in both experimental systems and in the clinic, even when all tumors harbor mutations that activate the pathways targeted by the drugs (Flaherty et al., 2010; Joseph et al., 2010; Pratilas et al., 2009; Slamon et al., 2001).

The MAPK signaling pathway is a main component in several steps of tumorigenesis including cancer cell proliferation, migration, invasion and survival. Overall, the activation of a MAPK employs a core three-kinase cascade. The extracellular mitogen binds to the membrane receptor (e.g., receptor tyrosine kinases, cytokine receptors, and some G protein-coupled receptors), which allows Ras (a GTPase) to swap its GDP for a GTP. It can now activate a MAPK kinase kinase (MAP3K or MAPKKK; e.g., Raf), which phosphorylates and activates a MAPK kinase (MAP2K, MEK, or MKK), which then phosphorylates and activates a MAPK (e.g., ERKs). Upon activation, MAPKs can phosphorylate and activate a variety of intracellular targets including transcription factors, nuclear pore proteins, membrane transporters, cytoskeletal elements, and other protein kinases.

The extracellular signal-regulated kinase (ERK) pathway (also referred to as the ERK-MAPK pathway, or the p44/42 MAPK pathway) is activated by a wide variety of mitogenic stimuli that interact with structurally distinct receptors and thus represents a convergence point for most, if not all, mitogenic signaling pathways (Seger R. et al., FASEB J., 1995, 9: 726-735; Lewis T. S. et al., Adv. Cancer Res., 1998, 74: 49-139; and Pearson G. et al., Endocr. Rev., 2001, 22: 153-183).

Mutations in signaling components that activate MAPKs have been found in many forms of cancer. Specifically, mutations in K-Ras are prominent in colon and pancreatic cancer; N-Ras and B-Raf mutations occur in melanomas; while H-Ras mutations are found in cervical and bladder cancer. At least 70% of melanoma tumors harbor an oncogenic mutation in the MAPK signaling pathway (Hodis et al., 2012).

Drugs targeting the MAPK signaling pathway have been recently approved with observed clinical success (Sosman et al., 2012). However, phenotypic responses to MAPK pathway inhibitors, both in patients and in vitro, vary significantly (Flaherty et al., 2010; Joseph et al., 2010).

The factors responsible for the response variability are largely unknown. Several molecular mechanisms have been proposed to explain response heterogeneity. Feedback reactivation of the pathway attenuates the inhibitory effects of the drugs. Different cell lines show different feedback dynamics (Lito et al., 2012; Poulikakos et al., 2010). Other studies found PTEN and MITF status correlated to response heterogeneity (Johannessen et al., 2013; Paraiso et al., 2011; Xing et al., 2012), but these explain only part of the observed variability.

A better understanding of the interactions and activity state of different pathways would enable clinicians to tailor new and unexpected drug combinations to individual patients, which may lead to better clinical responses.

### SUMMARY

The present invention provides for a method of treating cancer in a subject, comprising the step of administering to the subject an interferon and an inhibitor of mitogen-activated protein kinase (MAPK) signaling pathway, wherein the combination of the interferon and the inhibitor of the MAPK pathway produces a synergistic effect on the cancer compared to the effect of the interferon alone or the effect of the inhibitor of the MAPK pathway alone. The combination may result in a synergistic increase in apoptosis of cancer cells, and/or a synergistic reduction in tumor volume.

The present invention provides for a method of treating cancer in a subject, comprising the step of administering to the subject an interferon and a cytotoxic agent, wherein the combination of the interferon and the cytotoxic agent produces a synergistic effect on the cancer compared to the effect of the interferon alone or the effect of the cytotoxic agent alone. The combination may result in a synergistic increase in apoptosis of cancer cells, and/or a synergistic reduction in tumor volume.

The cytotoxic agent may be an inhibitor of MAPK signaling pathway, an alkylating agent, an anti-metabolite, an anti-microtubule agent, a topoisomerase inhibitor, a cytotoxic antibiotic, or an endoplasmic reticulum stress inducing agent.

Also encompassed by the present invention is a pharmaceutical composition comprising a first amount of an interferon and a second amount of an inhibitor of the mitogen-activated protein kinase (MAPK) signaling pathway, wherein the combination of the first amount of interferon and the second amount of the inhibitor of the MAPK pathway produces a synergistic effect on cancer compared to the effect of the first amount of interferon alone or the effect of the second amount of the inhibitor of the MAPK pathway alone. The combination may result in a synergistic increase in apoptosis of cancer cells, and/or a synergistic reduction in tumor volume.

The present invention provides for a method of treating cancer cells, comprising the steps of: (a) determining activity of STAT1 (Signal Transduction And Transcription 1) signaling pathway in the cancer cells; and (b) administering to the cancer cells an inhibitor of the mitogen-activated protein kinase (MAPK) signaling pathway, if the activity of the STAT1 signaling pathway in step (a) is less than 20% of activity of STAT1 signaling pathway in WM1361 melanoma cells. In step (b), an interferon may also be administered. In step (a), the activity of STAT signaling pathway may be determined by any of the following assays: assaying the level of pSTAT1-Y701 (STAT1 phosphorylated at Tyr701), an assay of protein level and/or phosphorylation level of JAK1/2, STAT1/2 and/or interferon receptors; an assay of expression levels of STAT1/2 downstream genes; and an assay of mRNA and/or protein levels of interferon-α or interferon-β.

The present invention also provides for a method of treating cancer cells, comprising the steps of: (a) determining copy number of interferon locus located on chromosome 9p22 in the cancer cells; (b) administering to the cancer cells an inhibitor of the mitogen-activated protein kinase (MAPK) signaling pathway, if the copy number of the interferon locus determined in step (a) is 0 or 1. In step (b), an interferon may also be administered.

The inhibitors of the MAPK pathway may be an inhibitor of RAF, an inhibitor of MEK, an inhibitor of ERK, an inhibitor of RAS, an inhibitor of receptor tyrosine kinases (RTKs), or combinations thereof. The inhibitor can be a small molecule, a polynucleotide (e.g., a small interfering RNA (siRNA) or an antisense molecule), a polypeptide, or an antibody or antigen-binding portion thereof. For example, the inhibitor is PLX4720, PD325901, GW5074, BAY 43-9006, ISIS 5132, PD98059, PD184352, U0126, Ro 09-2210, L-783,277, GSK-1120212, trametinic, vemurafenib, purvalanol, or imidazolium trans-imidazoledimethyl sulfoxide-tetrachlororuthenate (NAMI-A).

The interferon may be a type I, type II or type III interferon. Type I interferons include interferon-α, interferon-β, interferon-ε, interferon-κ, and interferon-ω.

The interferon and the inhibitor can be administered simultaneously, sequentially or separately.

The cancer that may be treated by the present methods and compositions include melanoma, breast cancer, colon cancer, pancreatic cancer, cervical cancer, thyroid cancer and bladder cancer.

### BRIEF DESCRIPTION OF THE DRAWINGS

FIG. 1—Phenotypic heterogeneity in response to MEK inhibition in melanoma. A. BRAF, NRAS, PTEN and MITF status show the genetic diversity of our panel of 14 cell line panel. We used 50 nM of PD325901 that fully inhibits the pathway in both NRAS and BRAF mutant cell lines (FIG. 9A). B. Percentage of TUNEL+ cells after 72 hours of treatment with DMSO (control) or PD325901 (50 nM). MAPK mutation, PTEN status and MITF status are listed at the bottom. C. Growth curves of untreated (control) and MEK-inhibited cells showing dramatically different responses. Cells were plated in 6-well plates, with 50K cells per well with 2 mL of growth media. Treated with DMSO (vehicle) or PD325901 after 24 h and counted every 24 hours.

FIG. 2—Transcriptional heterogeneity in response to MEK inhibition in melanoma. A. 3 gene clusters demonstrating the extent of context-specificity of MAPK targets. This, and all other Orange-Blue heat-maps in this manuscript, depicts the change in gene expression following treatment, with orange representing up-regulation and blue down-regulation. Rows represent genes and columns represent cell-lines. In each of the 3 clusters, cell lines show different response to MEK inhibition. Moreover, each cell line is unique and responses for each cell lines are different in each cluster. B. Number of differentially expressed genes as a function of fold change and number of cell lines. Arbitrarily choosing the cutoff is likely to mislabel hundreds of genes. BRAF and PTEN status are not correlated with transcriptional response to MEK inhibition (FIG. 10).

FIG. 3—COSPER identifies COntext-SPEcific Regulation—genes are under the control of MAPK in only a subset of cell lines, both before and after inhibition. A. We use HEY1 as an example for a context-specific target. HEY1 exhibits a context-specific behavior—regulated by MAPK in only a subset of cell lines (“Under MEK control”). MEK inhibition does not affect its expression in the other group of cells (“Not under MEK control”), and its basal expression is lower in these cell lines. B. A cartoon of context-specific regulation exhibited by HEY1. ERK up-regulates a set of targets only in genetic context 2, while it has no effect in the context 1 (upper panel). Therefore, the genes are down-regulated following MEK inhibition only in genetic context 2 (lower panel). C. COSPER identifies gene clusters with context-specific regulation. The cluster contains genes controlled by MAPK only in cell lines with high MITF mRNA expression. The Red-Green heat-map on the left shows basal expression levels (before pathway inhibition), with green representing low expression levels and red representing high levels. This and other red-green heat-maps compare expression levels between cell lines. The heat-map on the right shows fold change following MEK inhibition (comparing each cell line before and after inhibition). MITF expression, which is not part of this cluster, is in the top row. Several patterns of regulation (up- and down-regulations) are shown.

FIG. 4—Analysis of the clusters' genes allows facilitates the identification of pathways that exhibit context-specific interactions with the MAPK pathway. A. A cluster associated with MITF-M protein levels identified by COSPER. Its genes are overexpressed in high-MITF-M cell lines, and are down-regulated only in these cells after MEK inhibition. MITF expression is in the top row. The cluster is almost perfectly correlated with MITF-expression, except for one cell line highlighted in green. The binding site of MITF is overrepresented in the promoters of the cluster genes (p-value=10⁻³). Only part of this cluster's genes are shown (full cluster appears in FIG. 11A). B. MITF protein levels in all 14 cell lines. A2058 (rectangle) is the only low mRNA-MITF cell line that expresses the MITF-M isoform. C. Additional cluster identified by COSPER. The cluster's genes are enriched for STAT3-related GO annotations (full cluster appears in FIG. 11D). A bar indicating pSTAT3 levels appears in top row. D. As predicted by COSPER, pSTAT3-Y705 levels are correlated with the cluster. Cell lines with low-pSTAT3 are marked in red, matching the first 3 pSTAT3-low cell lines shown in C.

FIG. 5—IFNβ enhances cytotoxic response of MEK inhibition in low-pSTAT1 cell lines. A. COSPER identified a cluster containing several known interferon targets (marked in red). Three cell lines have high target expression, and MEK inhibition upregulates the pathway in the other 11 cell lines. A bar indicating pSTAT1 levels appears at the top and these are different than the high pSTAT3 cell lines of FIG. 4. B. pSTAT1-Y701, a marker for interferon-STAT1 pathway activity, is correlated with the gene expression and shows high basal activation level in the 3 high cell lines (WM1361, SkMel39, SkMel105). C. High interferon pathway activity is necessary, but not sufficient, for IFN-induced death. We used TUNEL staining as a marker for apoptosis 72 hours after IFNβ treatment. Only one out of 3 high-pSTAT1 cell lines respond to IFNβ and none of the low-PSTAT1 lines respond to IFNβ. We used IFNβ, and not IFNα, due to its higher efficacy (see FIG. 12A). D. MEK inhibition leads to up-regulation of pSTAT1 in all cell lines. E. MEK inhibition induces death in low-pSTAT1 cell lines only. IFNβ and MEK inhibition in low pSTAT1 cell lines synergize to increase apoptosis levels. High pSTAT1 cell lines show only mild response to the MEK inhibitor and its combination with IFNβ (right). IFNβ alone and untreated cells have almost no cytotoxic response.

FIG. 6—Elucidating the synergistic response of IFNβ and MEKi (MEK inhibitor). A. Response to IFNβ, as measured by pSTAT1 and IRF1 levels, is similar in both high- and low-pSTAT1 cell lines MEK inhibition does not alter the response (for transcriptional response see FIG. 13B). Notably, basal activity level of the pathway in high-pSTAT1 cell lines is much lower than the induction in pathway activity after IFNβ treatment. B. MEKi activates the intrinsic apoptotic pathway by cytochrome C release from the mitochondria, approx. 36 hours after treatment. IFNβ enhances the response in all cell lines, including the high-pSTAT1 resistant cell lines. C. Caspase 7 and 9 are cleaved and activated following MEK inhibition in low pSTAT1 cell lines only. IFNβ enhances MEKi's effect, but fails to activate the pathway by itself. Both caspases are not cleaved in high-pSTAT1 cell lines, explaining their resistance to treatment. To reinforce the association between STAT1 levels and response to MEK inhibition we tested 4 more cell lines. Both high- and low-pSTAT1 levels respond with accordance to their STAT1 levels (FIG. 13D).

FIG. 7—Deletion of interferon locus and IFN expression levels explains the two interferon-pathway states and predicts drug response. A. The interferon gene cluster identified by COSPER is highly correlated in the TCGA melanoma expression data set. This allows us to infer pathway activity in the TCGA tumors and associate it with DNA aberrations. Genes above the yellow line were used for association with DNA copy number. B. The interferon locus contains 17 interferon genes, and is only 0.5 Mb downstream of CDKN2A (p16), a known melanoma tumor suppressor. C. Interferon locus copy number is also correlated with pathway activity in our 14 cell line panel. p16 however, only 0.5 Mb upstream, is not, suggesting that interferon deletion and p16 deletion are two independent events. SkMel200, a high-pSTAT1 cell line, was added for purposes of CNV (copy number variation) analysis. Copy number of the interferon locus is also correlated with expression levels of interferon genes (FIG. 14B), and conditioned media experiment shows that cytokines are released from high pSTAT1 cell lines (FIG. 14C). D. A cartoon depicting the two network states, before and after MEKi and IFN treatment. Inhibition of MEK leads to cytochrome C release in both cellular contexts, and IFN treatment enhances the response. However, caspase 9 is cleaved and activated only in low pSTAT1 cell lines.

FIG. 8—FIG. 8A shows COSPER's target module. FIG. 8B shows COSPER's algorithm.

FIG. 9—A. pERK levels in all cell lines, 2, 4 and 8 hours following treatment with PD325901. pERK stays low throughput the 8 hours and therefore does not explain the heterogeneity observed between cell lines. B. Comparison of MEK and BRAF inhibitors in a BRAF-V600E cell line shows an almost identical transcriptional response. Scatter plot shows fold change of all genes with a 50 nM of PD325901, a MEK inhibitor (x-axis), compared with a 2 uM of PLX4720, a BRAF inhibitor (y-axis). Almost all genes fall directly on the diagonal. Colo829 doesn't grow in the conditions used to generate these growth curves. C-D. Scatter plots representation of the data in FIG. 1B. Each dot represents the difference of percentage of TUNEL+ cells between PD325901 and DMSO, dividing MITF+ and MITF− cell lines (B), and PTEN-WT and PTEN-null (C). These mutations fail to explain the phenotypic differences between cell lines.

FIG. 10—A. Histograms of p-values comparing expression levels of BRAF-mut with NRAS-mut cell lines using t-test, before and after pathway inhibition. No gene passes FDR correction with q-value<0.05. B. Histograms of p-values comparing expression levels of PTEN-null/mut with PTEN-WT in BRAF-mut cell lines using t-test, before and after pathway inhibition. No gene passes FDR correction with q-value<0.05.

FIG. 11—A. Full cluster, including all genes, of the cluster presented in FIG. 3A. B. MITF mRNA expression levels before (x-axis) and after (y-axis) MEK inhibition. Steady state and fold change levels are negatively correlated. C. Levels of MITF protein isoforms in 12 cell lines, before and 8 hours after MEK inhibition. Each isoform is regulated to different degrees in the different cell lines, supporting a context-specific control of MITF by the MAPK pathway. Strong (S) and Weak (W) film exposures are shown. D. COSPER clusters together genes with the same context-specific regulation but with different regulation patterns. For example, the cluster here is associated with the STAT3 context, but contains 3 regulation patterns. The cluster in FIG. 4C shows one such pattern out of the 3 identified by COSPER. E. Levels of STAT3 and pSTAT3-S727 are similar in all cell lines and do not explain the differential activation of pSTAT3-Y705.

FIG. 12—A. Dose-dependent response to IFNα and IFNβ. The cytotoxicity of IFN was assessed in high pSTAT1 cell line, 48 hours after treatment using SubG1 percentage. IFNα has a weaker cytotoxic effect than IFNβ, and both show dose-dependent effects. 1000 Units/mL of IFNβ was used for the experiments in Examples. Cells plated in 6 well plates, 200K cells/well, in 2 mL of growth media. B. Growth curves of 2 low- (top) and 2 high- (bottom) pSTAT1 cell lines with MEK inhibition, IFNβ or both. 50K cells per well were plated in 6-well plates with 2 mL of media and treated 24 hours later. Cells were counted every 24 hours up to 72 hours. C. Comparison of MEK inhibition and BRAF inhibition, with and without combination with IFNβ. Cells were plated in 6 well plates, 200K cells/well with 2 mL media. 24 hours after plating cells were treated with DMSO, 50 nM PD325901 with or without IFNβ, 2 μM of PLX4720 with or without IFNβ.

FIG. 13—A. Time course protein levels following treatment with IFNβ of one high pSTAT1 cell line (SkMel39) and one low (A375). Response amplitude and dynamics is almost identical in the two cell lines. B. 22 genes with the highest fold change following IFNβ treatment. The transcriptional response is similar in all cell lines, both with low- and high-basal activation of the pathway. Notably, the fold change of several genes reaches 100 fold, just 8 hours after treatment. C. Lack of synergistic and additive effects of MEK inhibition and IFNβ in gene expression. Scatter plots show the fold change of all genes with a combination of MEK inhibition and IFN (x-axis) and the sum of fold changes with each treatment alone. Significant deviations from the diagonal would demonstrate synergism between drugs. Only one gene, CCL4, deviates from the diagonal in all 6 cell lines. D. Cleaved caspases 9 and 3 following MEK inhibition, IFN treatment, or their combination. This figure includes 4 cell lines that were not part of the original caspase analysis, and were included here to support the association between pSTAT1 levels and activation of the caspase pathway. E. Caspase 9 and APAF1 levels (arrow marks APAF1 band) are not correlated with pSTAT1 levels or with cytotoxic response to MEK inhibition. F. Levels of known caspase inhibitors are not correlated with the cytotoxic phenotype or pSTAT1 levels.

FIG. 14—A. Protein levels following MEK inhibition of 6 known inhibitors of the JAK-STAT pathway in two cell lines (SkMel105—high pSTAT1, A375—low pSTAT1). Most proteins do not change, although pSTAT1 goes up prior to 8 h. Change of PIAS1 is similar in both cell lines. B. IFN genes with a significant differential expression between low- and high-pSTAT1 cell lines. IFNA6, IFNA8 and IFNB1 are located in the interferon locus. C. Conditioned media experiment shows that SkMel105, a high-pSTAT1 cell line, releases cytokines to the media that lead to the upregulation of pSTAT1. In this experiment, SkMel105 was cultured for 24 h, and then the media was transferred to A375, a low-pSTAT1 cell line. Cells were collected 30 m and 1 h following media transfer. Lanes for MEK inhibition 8 h and self-conditioned media (CM-A375) are shown as controls.

### DETAILED DESCRIPTION OF THE INVENTION

The present invention provides for methods and compositions for treating cancer. A subject having cancer is administered an interferon and an inhibitor of mitogen-activated protein kinase (MAPK) signaling pathway. The combination of the interferon and the inhibitor of the MAPK pathway produces a synergistic effect on the cancer compared to the effect of the interferon or the inhibitor of the MAPK pathway alone. For example, the combination may result in a synergistic increase in apoptosis of cancer cells, and/or a synergistic reduction in tumor volume.

The existing paradigm in MAPK pathway inhibition aims at a complete blocking of pro-survival signaling. Suggested combinatorial treatments include combinations of MAPK pathway inhibitors (such as RAF and MEK inhibitors (Corcoran et al., 2012)), or combinations that prevent the feedback activation of RTKs (Corcoran et al., 2012). However, the present examination of the pathway interactions and analysis of transcriptional response following MEK inhibition has identified a drug combination that takes a different approach. Instead of exerting all effort on shutting down MAPK signaling, we found that an interferon, which works via a different signaling pathway, enhances, e.g., the cytotoxicity of MAPK signaling inhibition.

Any component of the MAPK pathway may be inhibited by the present inhibitors. They include an inhibitor of RAF, an inhibitor of MEK, an inhibitor of MAPK (e.g., ERK), an inhibitor of RAS, an inhibitor of a receptor tyrosine kinase (RTK), or combinations thereof. The interferon (IFN) may be a type I, type II or type III interferon. Type I interferons include interferon-α, interferon-β, interferon-ε, interferon-κ, interferon-ω.

The present method for treating cancer may comprise the step of administering to a subject having cancer an interferon and a cytotoxic agent. The combination of the interferon and the cytotoxic agent produces a synergistic effect on the cancer compared to the effect of the interferon alone or the effect of the cytotoxic agent alone.

The present invention provides for a pharmaceutical composition comprising a first amount of an interferon and a second amount of an inhibitor of the mitogen-activated protein kinase (MAPK) signaling pathway. The combination of the first amount of interferon and the second amount of the inhibitor of the MAPK pathway produces a synergistic effect on cancer compared to the effect of the first amount of interferon alone or the effect of the second amount of the inhibitor of the MAPK pathway alone.

In another embodiment, the activity of the interferon pathway, interferon expression levels and/or interferon locus copy number can be used as biomarkers for treatment of cancer by MAPK pathway inhibitors.

Also encompassed by the present invention is a method for treating cancer cells. The method has the following steps: (a) determining activity of STAT1 (Signal Transduction And Transcription 1) signaling pathway in the cancer cells; and (b) administering to the cancer cells an inhibitor of the MAPK signaling pathway, if the activity of the STAT1 signaling pathway in step (a) is less than 20% of the activity of STAT1 signaling pathway in reference cells, e.g., WM1361 melanoma cells.

The present method of treating cancer cells may have the following steps: (a) determining copy number of interferon locus located on chromosome 9p22 in the cancer cells; (b) administering to the cancer cells an inhibitor of the mitogen-activated protein kinase (MAPK) signaling pathway, if the copy number of the interferon locus determined in step (a) is 0 or 1.

The present methods may be used in vitro or in a subject having cancer.

### Inhibitors of MAPK Signaling Pathway

Any component of the MAPK signaling pathway may be inhibited by the present inhibitors. They include an inhibitor of RAF, an inhibitor of MEK, an inhibitor of MAPK (ERK), an inhibitor of RAS, an inhibitor of a receptor tyrosine kinase (RTK), or combinations thereof.

Any isoform of any component the MAPK pathway may be inhibited by the present inhibitors. They include, but are not limited to: an inhibitor of BRAF, CRAF or ARAF; an inhibitor of MEK1, MEK2, MKK3, MKK4, MKK5, MKK6, or MKK7; an inhibitor of ERK1, ERK2, p38, JNK or ERK5; an inhibitor of HRAS, KRAS or NRAS; an inhibitor of epidermal growth factor receptor (EGFR), ErbB-2, ErbB-3, ErbB-4, Trk A/B, Fibroblast growth factor receptor (FGFR) or PDGFR.

The present inhibitors may target the wild-type or mutant component of the MAPK pathway. For example, the inhibitors may target, inhibit or decrease activity of wild-type BRAF or a mutant BRAF (e.g., BRAF(V600); BRAF(G466); BRAF(G464); BRAF(G469); BRAF(D594); BRAF(G596); BRAF(K601); BRAF(V600), etc.), wild-type MEK or a mutant MEK (e.g., MEK1/2(Q60), MEK1/2(P124), etc.), and wild-type RAS or a mutant RAS (e.g., N/K/H-RAS(Q61), N/K/H-RAS(G12), N/K/H-RAS(G13), etc.).

As used herein, the term “inhibitor” refers to agents capable of down-regulating or otherwise decreasing or suppressing the amount and/or activity of any component of the MAPK signaling pathway, including, but not limited to, the extracellular signal regulated mitogen-activated protein kinase (ERK-MAPK) signaling pathway.

The mechanism of inhibition may be at the genetic level (e.g., interference with or inhibit expression, transcription or translation, etc.) or at the protein level (e.g., binding, competition, etc.). The inhibitors may reduce MAPK signaling, reduce phosphorylation of components of the MAPK signaling pathways (e.g., MEK 1/2, ERK1/2), reduce levels of activated components of the MAPK signaling pathways (e.g., including but not limited to members of the Ras/Raf/MEK/ERK pathways), and/or sequester components of the MAPK signaling pathways and prevent signaling. For example, an inhibitor may be utilized that interferes with or inhibits expression of ERK1 and/or ERK2, or sequesters ERK 1 and/or ERK2 in the cytoplasm of the cell, preventing nuclear translocation and signaling (Brunet A. et al., EMBO J, 1999, 18: 664-674).

A wide variety of suitable inhibitors may be employed, guided by art-recognized criteria such as efficacy, toxicity, stability, specificity, half-life, etc.

### Small Molecule Inhibitors

As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules.

Non-limiting examples of MEK inhibitors include: PD325901, AZD6244 (Selumetinib; 6-(4-bromo-2-chloroanilino)-7-fluoro-N-(2-hydroxyethoxy)-3-methylbenzimidazole-5-carboxamide), R04987655, R05126766, TAK-733, MSC1936369B (AS703026), GSK1120212, BAY86-9766, GDC-0973, GDC-0623, ARRY-438162, 011040, E6201, ARRY300; PD98059, PD184352, U0126 (Dudley D. T. et al., Proc. Natl. Acad. Sci. USA, 1995, 92: 7686-7689; Sepolt-Leopold J. S. et al., Nat. Med., 1999, 5: 810-816; and Favata M. F. et al., J. Biol. Chem., 273: 18623-18632; Davies, S. P. et al., Biochem. J., 2000, 351: 95-105; Ahn N. G. et al., Methods Enzymol., 2001, 332: 417-431). A series of 3-cyano-4-(phenoxyanilo)quinolines with MEK inhibitory activity has also been developed by Wyeth-Ayerst (Zhang N. et al., Bioorg Med. Chem. Lett., 2000, 10: 2825-2828). Several resorcylic acid lactones having inhibitor activity toward MEK have been isolated. For example, Ro 09-2210, isolated from fungal broth FC2506, and L-783,277, purified from organic extracts of Phoma sp. (ATCC 74403), are competitive with ATP, and the MEK1 inhibition is reversible (Williams D. H. et al., Biochemistry, 1998, 37: 9579-9585; and Zhao A. et al., J. Antibiot., 1999, 52: 1086-1094). Imidazolium trans-imidazoledimethyl sulfoxide-tetrachlororuthenate (NAMI-A) is a ruthenium-containing inhibitor of the phosphorylation of MEK, the upstream activator of ERK (Pintus G. et al., Eur. J. Biochem., 2002, 269: 5861-5870).

Non-limiting examples of RAF inhibitors include: PLX4720; PLX4032 (Vemurafenib; N-(3-{[5-(4-chlorophenyl)-1H-pyrrolo[2,3-b]pyridin-3-yl]carbonyl}-2,4-difluorophenyl)propane-1-sulfonamide); R7204; GSK2118436; Sorafenib (BAY-43-9006); BMS-908662 (XL-281); RAF265 (Smalley and Flaherty (2009) Future Oncology, Volume 5, Number 6, pp. 775-778); RG-7256 (R05212054, PLX3603); R05126766; ARQ-736; E-3810; DCC-2036; 4-(4-{3-[4-chloro-3-(trifluoromethyl)phenyl]ureido}phenoxy)-N2-methylpyri-dine-2-carboxamide 4-methylbenzenesulfonate (sorafenib); GW5074; BAY 43-9006; and ISIS 5132 (Lackey, K. et al., Bioorg. Med. Chem. Lett., 2000, 10: 223-226; Lyons, J. F. et al., Endocrine-related Cancer, 2001, 8: 219-225; and Monia, B. P. et al., Nat. Med., 1996, 2(6): 668-675).

Non-limiting examples of ERK inhibitors include: GW5074, BAY 43-9006, ISIS 5132, PD98059, PD184352, U0126, Ro 09-2210, L-783,277, purvalanol (Knockaert M. et al., Oncogene, 2002, 21: 6413-6424), imidazolium trans-imidazoledimethyl sulfoxide-tetrachlororuthenate (NAMI-A), 3-cyano-4-(phenoxyanilno)quinolines (such as Wyeth-Ayerst Compound 14), resorcylic acid lactones (such as Ro 09-2210 and L-783,277), and purvalanol (Kohno M. et al., Progress in Cell Cycle Research, 2003, 5: 219-224). Information about ERK inhibitors and methods for their preparation are readily available in the art (see, for example, Kohno M. et al., Progress in Cell Cycle Research, 2003, 5: 219-224).

Non-limiting examples of p38 inhibitors include, RWJ 67657, SCIO 469, EO 1428, Org 48762-0, SD 169, SB 203580, SB 202190, SB 239063, SB 220025, VX-745, SB 242235, VX-702, SD-282, PH-797804, L-167307, RPR200765A, pamapimod, BIRB 796, BMS 582949, and others. See, e.g., Kumar et al., “p38 MAP Kinases: Key Signaling Molecules as Therapeutic Targets for Inflammatory Diseases,” Nature Reviews, 2:717-726 (2003); Brown et al., “p38 MAP kinase inhibitors as potential therapeutics for the treatment of joint degeneration and pain associated with osteoarthritis,” J. Inflammation 5:22 (2008); Mayer et al., “p38 MAP kinase inhibitors: A future therapy for inflammatory diseases,” Drug Discovery Today: Therapeutic Strategies 3(1): 49-54 (2006); and Regan et al., “Pyrazole Urea-Based Inhibitors of p38 MAP Kinase: from Lead Compound to Clinical Candidate,” J. Med. Chem. 2002, 45, 2994-3008, the entirety of each of which is incorporated herein by reference.

Non-limiting examples of receptor tyrosine kinases (RTKs) include inhibitors to ErbB: HER1/EGFR (Erlotinib, Gefitinib, Lapatinib, Vandetanib, Sunitinib, Neratinib); HER2/neu (Lapatinib, Neratinib); RTK class III: C-kit (Axitinib, Sunitinib, Sorafenib), FLT3 (Lestaurtinib), PDGFR (Axitinib, Sunitinib, Sorafenib); and VEGFR (Vandetanib, Semaxanib, Cediranib, Axitinib, Sorafenib); bcr-abl (Imatinib, Nilotinib, Dasatinib); Src (Bosutinib) and Janus kinase 2 (Lestaurtinib). The inhibitors also include lapatinib (Tykerb®); Zactima (ZD6474), Iressa (gefitinib), imatinib mesylate (STI571; Gleevec), erlotinib (OSI-1774; Tarceva), canertinib (CI 1033), semaxinib (SU5416), vatalanib (PTK787/ZK222584), sorafenib (BAY 43-9006), sutent (SUI 1248) and lefltmomide (SU101). PTK/ZK is a tyrosine kinase inhibitor with broad specificity that targets all VEGF receptors (VEGFR), the platelet-derived growth factor (PDGF) receptor, c-KIT and c-Fms. Drevs (2003) Idrugs 6(8):787-794. The chemical names of PTK/ZK are 1-[4-Chloroanilino]-4-[4-pyridylmethyl]phthalazine Succinate or 1-Phthalazinamine, N-(4-chlorophenyl)-4-(4-pyridinylmethyl)-butanedioate (1:1). Synonyms and analogs of PTK/TK are known as Vatalanib, CGP79787D, PTK787/ZK 222584, CGP-79787, DE-00268, PTK-787, PTK787A, VEGFR-TK inhibitor, ZK 222584 and ZK.

Inhibitors of the MAPK signaling pathway are also disclosed in U.S. Pat. Nos. 8,697,627 and 7,863,288; U.S. Patent Publication Nos. 2003/0060469; 2004/0048861; 2004/0082631; 2003-0232869; 20140275078, each of which is incorporated herein by reference in its entirety.

The structures of some inhibitors of the MAPK signaling pathway are shown below as examples.

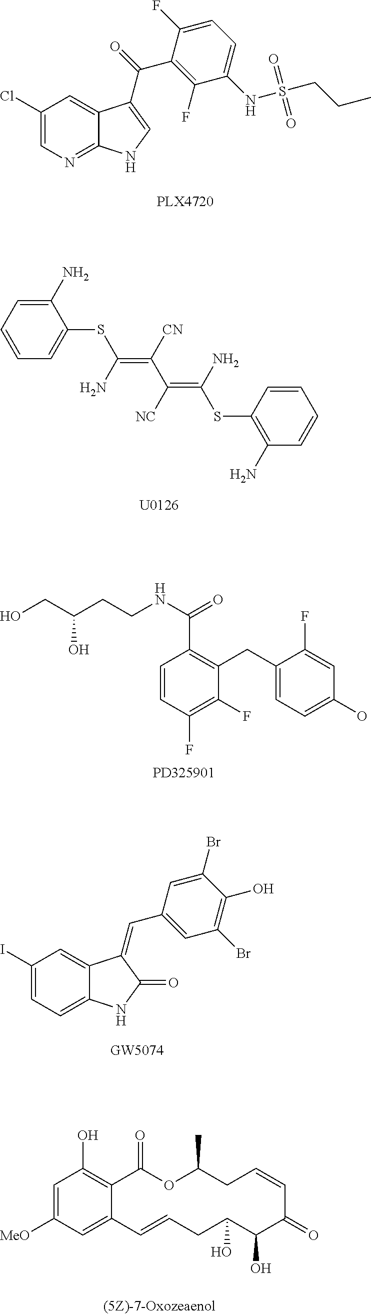 [SMILES: CCCS(=O)(=O)Nc1ccc(F)c(C(=O)c2c[nH]c3ncc(Cl)cc23)c1F ; N#CC(=C(/N)Sc1ccccc1N)/C(C#N)=C(\N)Sc1ccccc1N ; "U0126" ; O=C(NCC[C@H](O)CO)c1ccc(F)c(F)c1Cc1ccc(O)cc1F ; O=C1Nc2ccc(I)cc2/C1=C/c1cc(Br)c(O)c(Br)c1 ; COc1cc(O)c2c(c1)/C=C/C[C@H](O)[C@H](O)C(=O)/C=C\C[C@H](C)OC2=O]

In certain embodiments, the MAPK pathway inhibitor used in the methods and compositions of the invention is a polynucleotide that reduces expression of one or more components of the MAPK pathway. Thus, the method involves administering an effective amount of a polynucleotide that specifically targets nucleotide sequence(s) within a target gene(s) of the MAPK pathways. The polynucleotides reduce expression of one or more genes within the MAPK pathways, to yield reduced levels of the gene product (the translated polypeptide).

The nucleic acid target of the polynucleotides (e.g., siRNA, antisense oligonucleotides, and ribozymes) of the invention may be any location within the gene or transcript of any component of the MAPK signaling pathway.

### RNA Interference

SiRNAs (small interfering RNAs) or small-hairpin RNA (shRNA) may be used to reduce the level of any component of the MAPK signaling pathway.

SiRNAs may have 16-30 nucleotides, e.g., 16, 17, 18, 19, 20, 21, 22, 23, 24, 25, 26, 27, 28, 29, or 30 nucleotides. The siRNAs may have fewer than 16 or more than 30 nucleotides. The polynucleotides of the invention include both unmodified siRNAs and modified siRNAs such as siRNA derivatives etc.

SiRNAs can be delivered into cells in vitro or in vivo by methods known in the art, including cationic liposome transfection and electroporation. SiRNAs and shRNA molecules can be delivered to cells using viruses or DNA vectors.

### Antisense Polynucleotides

In other embodiments, the polynucleotide of the invention is an antisense nucleic acid sequence that is complementary to a target region within the mRNA of any component of the MAPK signaling pathway. The antisense polynucleotide may bind to the target region and inhibit translation. The antisense oligonucleotide may be DNA or RNA, or comprise synthetic analogs of ribo-deoxynucleotides. Thus, the antisense oligonucleotide inhibits expression of any component of the MAPK signaling pathway.

An antisense oligonucleotide can be, for example, about 7, 10, 15, 20, 25, 30, 35, 40, 45, 50, 55, 60, 65, 70, 75, 80, or more nucleotides in length.

An example of an antisense oligonucleotide with inhibitory activity toward ERK signaling is ISIS 5132, a 20-base phosphorothioate antisense oligodoxynucleotide designed to hybridize to the 3′ untranslated region of the c-raf-1 mRNA (Monia, B. P. et al., Nat. Med., 1996, 2(6): 668-675; Stevenson J. P. et al., J. Clin. Oncol., 1999, 17: 2227-2236; O'Dwyer P. J. et al., Clin. Cancer Res., 1999, 5: 3977-3982). Inhibition of ERK can also employ approaches disclosed in Pages G. et al., Proc. Natl. Acad. Sci. USA, 1993, 90: 8319-8323.

The antisense nucleic acid molecules of the invention may be administered to a subject, or generated in situ such that they hybridize with or bind to the mRNA of a component of the MAPK signaling pathway. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using viruses or DNA vectors.

### Ribozyme

In other embodiments, the polynucleotide of the invention is a ribozyme that inhibits expression of the gene of any component of the MAPK signaling pathway.

Ribozymes can be chemically synthesized in the laboratory and structurally modified to increase their stability and catalytic activity using methods known in the art. Alternatively, ribozyme encoding nucleotide sequences can be introduced into host cells through gene-delivery mechanisms known in the art. U.S. Pat. Nos. 8,592,368 and 5,093,246. Haselhoff et al., Nature 334: 585-591 (1988).

Other aspects of the invention include vectors (e.g., viral vectors, expression cassettes, plasmids) comprising or encoding polynucleotides of the subject invention (e.g., siRNA, antisense nucleic acids, and ribozymes), and host cells genetically modified with polynucleotides or vectors of the subject invention.

### Polypeptides

The present inhibitors can also be a polypeptide exhibiting inhibitory activity toward any component of the MAPK signaling pathway. For example, a receptor decoy may be used. A peptide corresponding to the amino-terminal 13 amino acids of MEK1 (MPKKKPTPIQLNP; SEQ ID NO: 1), can be used to inhibit the activation of ERK1/2 (Kelemen B. R. et al., J. Biol. Chem., 2002, 277: 87841-8748).

Various means for delivering polypeptides to a cell can be utilized to carry out the methods of the subject invention. For example, protein transduction domains (PTDs) can be fused to the polypeptide, producing a fusion polypeptide, in which the PTDs are capable of transducing the polypeptide cargo across the plasma membrane (Wadia, J. S. and Dowdy, S. F., Curr. Opin. Biotechnol., 2002, 13(1)52-56).

According to the methods of the subject invention, recombinant cells can be administered to a patient, wherein the recombinant cells have been genetically modified to express a nucleotide sequence encoding an inhibitory polypeptide.

### Antibodies

The present inhibitors can be an antibody or antigen-binding portion thereof that is specific to any component of the MAPK signaling pathway, thereby inhibiting the MAPK signaling.

The antibody or antigen-binding portion thereof may be the following: (a) a whole immunoglobulin molecule; (b) an scFv; (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human antibodies.

### Interferons

Interferons encompasses type I, type II and type III interferons. The interferon may be a human interferon.

Type I interferons include interferon-α, interferon-β, interferon-ε, interferon-κ, and interferon-ω. Type II interferons include interferon-γ. Type III interferons include interferon-λ.

The interferon used in the present methods and compositions may a peptide or protein having an amino acid sequence substantially identical (e.g., at least 70%, at least 75%, at least 80%, at least 85%, at least 90%, at least 95%, at least 96%, at least 97%, at least 98%, at least 99%, or 100% identical) to all or a portion of the sequence of a wild-type interferon. U.S. Patent Application Nos. 20070274950; 20040247565 and 20070243163; U.S. Pat. Nos. 7,238,344; 6,962,978; 4,588,585; 4,959,314; 4,737,462; 4,450,103; 5,738,845; and PCT Publication No. WO 07/044,083, each of which is incorporated by reference in their entirety.

The interferons may also be modified, such as PEGylated interferons (PEG-IFNs). The interferons used in the present methods and compositions also include variants of interferons such as fragments, consensus interferons (CIFNs), interferons with altered glycosylation (non-native glycosylation or aglycosylated), non-natural interferons, recombinant interferons, interferon mutants. Those skilled in the art are well aware of different interferons including those that are commercially available and in use as therapeutics.

The biological activity of an interferon of the invention can be confirmed using, e.g., a virus-plaque-reduction assay, assays that measure the inhibition of cell proliferation, the regulation of functional cellular activities, the regulation of cellular differentiation, and immunomodulation mediated by IFN, as well as a reporter gene assay, in which the promoter region of IFN responsive genes is linked with a heterologous reporter gene, for example, firefly luciferase or alkaline phosphatase, and transfected into an IFN-sensitive cell line such that stably transfected cell lines exposed to IFN increase expression of the reporter gene product in direct relation to the dose of IFN (see, e.g., Balducci et al., Appl. Microbiol. 11:310-314, 1963; McNeil, J. Immunol. Methods 46:121-127, 1981; and Meager et al., J. Immunol. Methods 261:21-36, 2002). Other assays for measuring the activity of IFN include measuring the up-regulation or activity of the double-stranded RNA (dsRNA)-dependent protein kinase R (PKR), the 2′-5′-oligoadenylate synthetase (2′-5′-OAS), IFN-inducible Mx proteins, a tryptophan-degrading enzyme (see, e.g., Pfefferkorn, Proc. Natl. Acad. Sci. USA 81:908-912, 1984), adenosine deaminase (ADAR1), IFN-stimulated gene 20 (ISG20), p 56, ISG15, mGBP2, GBP-1, the APOBEC proteins, viperin, or other factors (see, e.g., Zhang et al., J. Virol., 81:11246-11255, 2007, and U.S. Pat. No. 7,442,527, which are incorporated by reference herein in their entirety).

Interferons may be synthetic, recombinant or purified. Interferons can also be expressed using a vector that includes a nucleic acid sequence encoding the interferon.

### Combination Therapy

The present method for treating cancer may comprise the step of administering to a subject an interferon and an inhibitor of the MAPK signaling pathway.

This may be achieved by administering a pharmaceutical composition that includes both agents (an interferon and an inhibitor of the MAPK signaling pathway), or by administering two pharmaceutical compositions, at the same time or within a short time period, wherein one composition comprises an interferon, and the other composition includes an inhibitor of the MAPK signaling pathway.

The combination of the interferon and the inhibitor of the MAPK pathway produces an additive or synergistic effect (i.e., greater than additive effect) in treating the cancer compared to the effect of the interferon or the inhibitor of the MAPK pathway alone. For example, the combination may result in a synergistic increase in apoptosis of cancer cells, and/or a synergistic reduction in tumor volume. In different embodiments, depending on the combination and the effective amounts used, the combination of compounds can inhibit tumor growth, achieve tumor stasis, or achieve substantial or complete tumor regression.

In various embodiments, the present invention provides methods to reduce cancer cell growth, proliferation, and/or metastasis, as measured according to routine techniques in the diagnostic art. Specific examples of relevant responses include reduced size, mass, or volume of a tumor, or reduction in cancer cell number.

The present compositions and methods can have one or more of the following effects on cancer cells or the subject: cell death; decreased cell proliferation; decreased numbers of cells; inhibition of cell growth; apoptosis; necrosis; mitotic catastrophe; cell cycle arrest; decreased cell size; decreased cell division; decreased cell survival; decreased cell metabolism; markers of cell damage or cytotoxicity; indirect indicators of cell damage or cytotoxicity such as tumor shrinkage; improved survival of a subject; preventing, inhibiting or ameliorating the cancer in the subject, such as slowing progression of the cancer, reducing or ameliorating a sign or symptom of the cancer; reducing the rate of tumor growth in a patient; preventing the continued growth of a tumor, reducing the size of a tumor; and/or disappearance of markers associated with undesirable, unwanted, or aberrant cell proliferation. U.S. Patent Publication No. 20080275057 (incorporated herein by reference in its entirety).

Methods and compositions of the present invention can be used for prophylaxis as well as amelioration of signs and/or symptoms of cancer.

In some embodiments, the combination therapy results in a synergistic effect, for example, the interferon and the inhibitor of the MAPK pathway act synergistically, for example, in the apoptosis of cancer cells, inhibition of proliferation/survival of cancer cells, in the production of tumor stasis.

As used herein, the term “synergy” (or “synergistic”) means that the effect achieved with the methods and combinations of this invention is greater than the sum of the effects that result from using the individual agents alone, e.g., using the interferon alone and the inhibitor of the MAPK pathway alone. For example, the effect (e.g., apoptosis of cells, a decrease in cell viability, cytotoxicity, a decrease in cell proliferation, a decrease in cell survival, inhibition of tumor growth, a reduction in tumor volume, and/or tumor stasis, etc. as described herein) achieved with the combination of an interferon and an inhibitor of the MAPK pathway is about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 8 fold, about 9 fold, about 10 fold, about 12 fold, about 15 fold, about 20 fold, about 25 fold, about 30 fold, about 50 fold, about 100 fold, at least about 1.2 fold, at least about 1.5 fold, at least about 2 fold, at least about 2.5 fold, at least about 3 fold, at least about 3.5 fold, at least about 4 fold, at least about 4.5 fold, at least about 5 fold, at least about 5.5 fold, at least about 6 fold, at least about 6.5 fold, at least about 7 fold, at least about 8 fold, at least about 9 fold, at least about 10 fold, of the sum of the effects that result from using the interferon alone and the inhibitor of the MAPK pathway alone.

Synergistic effects of the combination may also be evidenced by additional, novel effects that do not occur when either agent is administered alone, or by reduction of adverse side effects when either agent is administered alone.

Cytotoxicity effects can be determined by any suitable assay, including, but not limited to, assessing cell membrane integrity (using, e.g., dyes such as trypan blue or propidium iodide, or using lactate dehydrogenase (LDH) assay), measuring enzyme activity, measuring cell adherence, measuring ATP production, measuring co-enzyme production, measuring nucleotide uptake activity, crystal violet method, Tritium-labeled Thymidine uptake method, measuring lactate dehydrogenase (LDH) activity, 3-(4,5-Dimethyl-2-thiazolyl)-2,5-diphenyl-2H-tetrazolium bromide (MTT) or MTS assay, sulforhodamine B (SRB) assay, WST assay, clonogenic assay, cell number count, monitoring cell growth, etc.

Apoptosis of cells may be assayed by any suitable method, including, but not limited to, TUNEL (terminal deoxynucleotidyl transferase dUTP nick end labeling) assay, assaying levels of cytochrome C release, assaying levels of cleaved/activated caspases, assaying 5-bromo-2′-deoxyuridine labeled fragmented DNA, assaying levels of survivin etc.

Other methods that can be used to show the synergistic effects of the present methods, pharmaceutical compositions and combinations include, but are not limited to, clonogenic assay (colony formation assay) to show decrease in cell survival and/or proliferation, studying tumor volume reduction in animal models (such as in mice, etc.)

In one embodiment, advantageously, such synergy provides greater efficacy at the same doses, lower side effects, and/or prevents or delays the build-up of multi-drug resistance.

The interferon and the inhibitor of the MAPK signaling pathway may be administered simultaneously, separately or sequentially. They may exert an advantageously combined effect (e.g., additive or synergistic effects).

For sequential administration, either an interferon is administered first and then an MAPK pathway inhibitor, or the MAPK pathway inhibitor is administered first and then an interferon. In embodiments where interferon and an inhibitor of the MAPK signaling pathway are administered separately, administration of a first agent can precede administration of a second agent by seconds, minutes, hours, days, or weeks. The time difference in non-simultaneous administrations may be greater than 1 minute, and can be, for example, precisely, at least, up to, or less than 5 minutes, 10 minutes, 15 minutes, 30 minutes, 45 minutes, 60 minutes, 2 hours, 3 hours, 6 hours, 9 hours, 12 hours, 24 hours, 36 hours, or 48 hours, or more than 48 hours. The two or more agents can be administered within minutes of each other or within about 0.5, about 1, about 2, about 3, about 4, about 6, about 9, about 12, about 15, about 18, about 24, or about 36 hours of each other or within about 1, 2, 3, 4, 5, 6, 7, 8, 9, 10, 12, 14 days of each other or within about 2, 3, 4, 5, 6, 7, 8, 9, or 10 weeks of each other. In some cases longer intervals are possible.

The present invention also provides for a pharmaceutical composition comprising (i) an interferon; (ii) an inhibitor of the MAPK signaling pathway; and (iii) at least one pharmaceutically acceptable excipient.

### Cytotoxic Agents

The present method for treating cancer may comprise the step of administering to a subject having cancer an interferon and a cytotoxic agent. The combination of the interferon and the cytotoxic agent produces a synergistic effect on the cancer compared to the effect of the interferon alone or the effect of the cytotoxic agent alone. The synergist effects are discussed herein.

The cytotoxic agent may be any chemotherapeutic agents including, but not limited to, alkylating agents, anti-metabolites, anti-microtubule agents, topoisomerase inhibitors, cytotoxic antibiotics, endoplasmic reticulum stress inducing agents, platinum compounds, vincalkaloids, taxanes, epothilones, enzyme inhibitors, receptor antagonists, tyrosine kinase inhibitors, boron radiosensitizers (i.e. velcade), and chemotherapeutic combination therapies.

Non-limiting examples of DNA alkylating agents are nitrogen mustards, such as Cyclophosphamide (Ifosfamide, Trofosfamide), Chlorambucil (Melphalan, Prednimustine), Bendamustine, Uramustine and Estramustine; nitrosoureas, such as Carmustine (BCNU), Lomustine (Semustine), Fotemustine, Nimustine, Ranimustine and Streptozocin; alkyl sulfonates, such as Busulfan (Mannosulfan, Treosulfan); Aziridines, such as Carboquone, Triaziquone, Triethylenemelamine; Hydrazines (Procarbazine); Triazenes such as Dacarbazine and Temozolomide (TMZ); Altretamine and Mitobronitol.

Non-limiting examples of Topoisomerase I inhibitors include Campothecin derivatives including SN-38, APC, NPC, campothecin, topotecan, exatecan mesylate, 9-nitrocamptothecin, 9-aminocamptothecin, lurtotecan, rubitecan, silatecan, gimatecan, diflomotecan, extatecan, BN-80927, DX-8951f, and MAG-CPT as decribed in Pommier Y. (2006) Nat. Rev. Cancer 6(10):789-802 and U.S. Patent Publication No. 200510250854; Protoberberine alkaloids and derivatives thereof including berberrubine and coralyne as described in Li et al. (2000) Biochemistry 39(24):7107-7116 and Gatto et al. (1996) Cancer Res. 15(12):2795-2800; Phenanthroline derivatives including Benzo[i]phenanthridine, Nitidine, and fagaronine as described in Makhey et al. (2003) Bioorg. Med. Chem. 11 (8): 1809-1820; Terbenzimidazole and derivatives thereof as described in Xu (1998) Biochemistry 37(10):3558-3566; and Anthracycline derivatives including Doxorubicin, Daunorubicin, and Mitoxantrone as described in Foglesong et al. (1992) Cancer Chemother. Pharmacol. 30(2):123-125, Crow et al. (1994) J. Med. Chem. 37(19):31913194, and Crespi et al. (1986) Biochem. Biophys. Res. Commun. 136(2):521-8. Topoisomerase II inhibitors include, but are not limited to Etoposide and Teniposide. Dual topoisomerase I and II inhibitors include, but are not limited to, Saintopin and other Naphthecenediones, DACA and other Acridine-4-Carboxamindes, Intoplicine and other Benzopyridoindoles, TAS-I03 and other 7H-indeno[2,1-c]Quinoline-7-ones, Pyrazoloacridine, XR 11576 and other Benzophenazines, XR 5944 and other Dimeric compounds, 7-oxo-7H-dibenz[f,ij]Isoquinolines and 7-oxo-7H-benzo[e]pyrimidines, and Anthracenyl-amino Acid Conjugates as described in Denny and Baguley (2003) Curr. Top. Med. Chem. 3(3):339-353. Some agents inhibit Topoisomerase II and have DNA intercalation activity such as, but not limited to, Anthracyclines (Aclarubicin, Daunorubicin, Doxorubicin, Epirubicin, Idarubicin, Amrubicin, Pirarubicin, Valrubicin, Zorubicin) and Antracenediones (Mitoxantrone and Pixantrone).

Examples of endoplasmic reticulum stress inducing agents include, but are not limited to, dimethyl-celecoxib (DMC), nelfinavir, celecoxib, and boron radiosensitizers (i.e. velcade (Bortezomib)).

Platinum based compounds are a subclass of DNA alkylating agents. Non-limiting examples of such agents include Cisplatin, Nedaplatin, Oxaliplatin, Triplatin tetranitrate, Satraplatin, Aroplatin, Lobaplatin, and JM-216. (See McKeage et al. (1997) J. Clin. Oncol. 201:1232-1237 and in general, CHEMOTHERAPY FOR GYNECOLOGICAL NEOPLASM, CURRENT THERAPY AND NOVEL APPROACHES, in the Series Basic and Clinical Oncology, Angioli et al. Eds., 2004).

“FOLFOX” is an abbreviation for a type of combination therapy that is used to treat colorectal cancer. It includes 5-FU, oxaliplatin and leucovorin.

“FOLFOX/BV” is an abbreviation for a type of combination therapy that is used to treat colorectal cancer. This therapy includes 5-FU, oxaliplatin, leucovorin and Bevacizumab. Furthennore, “XELOX/BV” is another combination therapy used to treat colorectal cancer, which includes the prodrug to 5-FU, known as Capecitabine (Xeloda) in combination with oxaliplatin and bevacizumab.

Non-limiting examples of antimetabolite agents include Folic acid based, i.e. dihydrofolate reductase inhibitors, such as Aminopterin, Methotrexate and Pemetrexed; thymidylate synthase inhibitors, such as Raltitrexed, Pemetrexed; Purine based, i.e. an adenosine deaminase inhibitor, such as Pentostatin, a thiopurine, such as Thioguanine and Mercaptopurine, a halogenated/ribonucleotide reductase inhibitor, such as Cladribine, Clofarabine, Fludarabine, or a guanine/guanosine: thiopurine, such as Thioguanine; or Pyrimidine based, i.e. cytosine/cytidine: hypomethylating agent, such as Azacitidine and Decitabine, a DNA polymerase inhibitor, such as Cytarabine, a ribonucleotide reductase inhibitor, such as Gemcitabine, or a thymine/thymidine: thymidylate synthase inhibitor, such as a Fluorouracil (5-FU). Equivalents to 5-FU include prodrugs, analogs and derivative thereof such as 5′-deoxy-5-fluorouridine (doxifluroidine), 1-tetrahydrofuranyl-5-fluorouracil (ftorafur), Capecitabine (Xeloda), S-I (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamicheal (1999) The Oncologist 4:478-487.

Examples of vincalkaloids, include, but are not limited to Vinblastine, Vincristine, Vinflunine, Vindesine and Vinorelbine.

Examples of taxanes include, but are not limited to docetaxel, Larotaxel, Ortataxel, Paclitaxel and Tesetaxel. An example of an epothilone is iabepilone.

Examples of enzyme inhibitors include, but are not limited to farnesyltransferase inhibitors (e.g., Tipifarnib); CDK inhibitors (e.g., Alvocidib, Seliciclib); proteasome inhibitors (e.g., Bortezomib); phosphodiesterase inhibitors (e.g., Anagrelide; rolipram); IMP dehydrogenase inhibitors (e.g., Tiazofurine); and lipoxygenase inhibitors (e.g., Masoprocol).

Chemotherapeutic agents may also include amsacrine, Trabectedin, retinoids (Alitretinoin, Tretinoin), Arsenic trioxide, asparagine depleter Asparaginase/Pegaspargase), Celecoxib, Demecolcine, Elesclomol, Elsamitrucin, Etoglucid, Lonidamine, Lucanthone, Mitoguazone, Mitotane, Oblimersen, Temsirolimus, and Vorinostat.

### Conditions to be Treated

Cancers treated using methods and compositions described herein are characterized by abnormal cell proliferation including, but not limited to, pre-neoplastic hyperproliferation, cancer in-situ, neoplasms and metastasis.

Cancers that can be treated by the present compositions and methods include, but are not limited to, melanoma, breast cancer, colorectal cancer, pancreatic cancer, cervical cancer, thyroid cancer, bladder cancer, non-small cell lung cancer, liver cancer, prostate cancer, muscle cancer, hematological malignancies, endometrial cancer, lymphomas, sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, synovioma, mesothelioma, lymphangioendotheliosarcoma, Ewing's tumor, leiomyosarcoma, rhabdomyosarcoma, colon carcinoma, ovarian cancer, gastric cancer, esophageal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, non-small cell lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, ear, nose and throat cancer, hematopoietic cancer, biliary tract cancer; bladder cancer; bone cancer; choriocarcinoma; connective tissue cancer; cancer of the digestive system; esophageal cancer; eye cancer; cancer of the head and neck; gastric cancer; intra-epithelial neoplasm; kidney cancer; larynx cancer; leukemia including acute myeloid leukemia, acute lymphoid leukemia, chronic myeloid leukemia, chronic lymphoid leukemia; lymphoma including Hodgkin's and Non-Hodgkin's lymphoma; myeloma; fibroma, oral cavity cancer (e.g., lip, tongue, mouth, and pharynx); prostate cancer; retinoblastoma; rhabdomyosarcoma; rectal cancer; renal cancer; cancer of the respiratory system; skin cancer; stomach cancer; testicular cancer; uterine cancer; cancer of the urinary system, as well as other carcinomas and sarcomas. U.S. Pat. No. 7,601,355.

The present invention also provides methods of treating neurological disorders, including, but not limited to, cerebral ischemia, Alzheimer's disease or Parkinson's disease.

The present compositions may be administered alone, or in combination with radiation, surgery or chemotherapeutic agents. The present compositions may be administered before, during or after the administration of radiation, surgery or chemotherapeutic agents.

### Tailored Cancer Therapy

Cancer cells may be treated by the following method: (a) determining activity of STAT1 (Signal Transduction And Transcription 1) signaling pathway in the cancer cells; and (b) administering to the cancer cells an inhibitor of the MAPK signaling pathway, if the activity of the STAT1 signaling pathway in step (a) is less than 60%, less than 50%, less than 40%, less than 30%, less than 20%, less than 10%, less than 7%, less than 5%, less than 3%, less than 1%, of the activity of STAT1 signaling pathway in reference cells, e.g., WM1361 melanoma cells. The activity of the STAT1 signaling pathway in the cancer cells may be extremely low or undetectable.

Also encompassed by the present invention is a method for treating cancer in a subject. The method has the following steps: (a) determining activity of STAT1 (Signal Transduction And Transcription 1) signaling pathway in the cancer cells; and (b) administering to the subject an inhibitor of the MAPK signaling pathway, if the activity of the STAT1 signaling pathway in step (a) is less than 60%, less than 50%, less than 40%, less than 30%, less than 20%, less than 10%, less than 7%, less than 5%, less than 3%, less than 1%, of the activity of STAT1 signaling pathway in reference cells, e.g., WM1361 melanoma cells.

In step (b) of the above methods, an interferon may also be administered. Besides WM1361 melanoma cells, SkMel39 melanoma cells and other cell lines with high activity of the STAT1 signaling pathway may alternatively be used as the reference cells in step (b).

In step (a) of the above methods, the activity of the STAT1 signaling pathway can be determined by assaying the level of pSTAT1-Y701 (STAT1 phosphorylated at Tyr701). The activity of the STAT1 signaling pathway may also be determined by any of the following assay, or a combination thereof: (i) an assay of gene expression signature; (ii) an assay of protein level or phosphorylation level of JAK1/2, STAT1/2 and/or interferon receptors; (iii) an assay of expression levels of STAT1/2 downstream genes; and (iv) an assay of mRNA and protein levels of interferon-α or interferon-β.

Protein phosphorylation (or level of the phosphorylated protein) may be measured by any suitable assays, including, but not limiting to, colorimetric, chemiluminescent, radioactive or fluorometric detection methods. Phosphorylation-specific antibodies (i.e., antibodies specific to the phosphorylated protein) may be used in combination with Western blot, enzyme-linked immunosorbent assay (ELISA), flow cytometry, immunocytochemistry or immunohistochemistry. Mass spectrometry (MS) may also be used to assess protein phosphorylation. Several enrichment strategies for phospho-protein analysis by MS have been developed including immobilized metal affinity chromatography (IMAC), phosphor-specific antibody enrichment, chemical-modification-based methods such as beta-elimination of phospho-serine and phospho-threonine, and replacement of the phosphate group with biotinylated moieties. Protein phosphorylation may also be assayed using 2-dimensional gel electrophoresis.

The level of a protein may be determined by any suitable assays, including, but not limited to, using antibodies specific to the protein in Western blot, enzyme-linked immunosorbent assay (ELISA), flow cytometry, immunocytochemistry or immunohistochemistry.

Gene expression levels may be assayed by any suitable methods, including, but not limited to, measuring mRNA levels and/or protein levels. Levels of mRNA can be quantitatively measured by RT-PCR, Northern blot, next-generation sequencing, etc.

A gene expression signature is a group of genes in a cell whose combined expression pattern is uniquely characteristic of a signaling pathway, a biological phenotype, a medical condition, etc.

In another embodiment, the present method of inhibiting proliferation of cancer cells may have the following steps: (a) determining copy number of interferon locus located on chromosome 9p22 in the cancer cells; (b) administering to the cancer cells an inhibitor of the mitogen-activated protein kinase (MAPK) signaling pathway, if the copy number of the interferon locus determined in step (a) is 0 or 1. In step (b), an interferon may also be administered.

Similarly, cancer in a subject may be treated using the following method: (a) determining copy number of interferon locus located on chromosome 9p22 in the cancer cells; (b) administering to the subject an inhibitor of the mitogen-activated protein kinase (MAPK) signaling pathway, if the copy number of the interferon locus determined in step (a) is 0 or 1. In step (b) of the above methods, an interferon may also be administered.

Various assays may be used to assess DNA copy numbers, including, but not limited to, comparative genomic hybridization (CGH), array CGH (aCGH) (Chung et al. (2004) “A whole-genome mouse BAC microarray with 1-Mb resolution for analysis of DNA copy number changes by array comparative genomic hybridization.” Genome research 14, 188-196. Liang et al. (2008) “Extensive genomic copy number variation in embryonic stem cells.” Proceedings of the National Academy of Sciences of the United States of America 105, 17453-17456). More detailed CGH may also be used. For example, an oligo aCGH platform (Agilent Technologies) not only enables one to study genome-wide DNA copy number at high resolution (Barrett et al. (2004). “Comparative genomic hybridization using oligonucleotide microarrays and total genomic DNA.” Proceedings of the National Academy of Sciences of the United States of America 101, 17765-17770.), but permit examination of a specific genome region using custom designed arrays. 1M SurePrint CGH arrays (Agilent Technologies) or chromosomal microarray may also be used to assess DNA copy numbers. U.S. Patent Publication No. 20140283150.

DNA copy number can also be detected using, for example, fluorescence in situ hybridization (FISH), non-fluorescent ISH (e.g., bright-field ISH), combined binary ratio labeling FISH (COBRA-FISH), spectral karyotyping (SKY), flow-FISH, Fiber-FISH (FISH to DNA fibers), Giemsa banding (G-banding), Q-banding, C-banding, R-banding, whole chromosome painting (WCP), and other cytogenetic techniques.

DNA copy number may be assayed by Southern blotting, PCR (polymerase chain reaction), quantitative PCR, quantitative real time PCR (qPCR), quantitative fluorescence PCR (QF-PCR), digital PCR, 3D digital PCR, multiplex ligation-dependent probe amplification (MLPA), next-generation sequencing (e.g., massively parallel signature sequencing (MPSS), polony sequencing, 454 pyrosequencing, Illumina (Solexa) sequencing, SOLiD sequencing, Ion Torrent semiconductor sequencing, DNA nanoball sequencing, Heliscope single molecule sequencing, Single molecule real time (SMRT) sequencing), etc.

### Pharmaceutical Compositions

The present invention provides for a pharmaceutical composition comprising a first amount of an interferon and a second amount of an inhibitor of the mitogen-activated protein kinase (MAPK) signaling pathway. The combination of the first amount of interferon and the second amount of the inhibitor of the MAPK pathway produces a synergistic effect on cancer (or in treating other disorders) compared to the effect of the first amount of interferon alone or the effect of the second amount of the inhibitor of the MAPK pathway alone.

The amount of interferon or the amount of the inhibitor of the mitogen-activated protein kinase (MAPK) signaling pathway that may be used in the combination therapy may be a therapeutically effective amount, a sub-therapeutically effective amount or a synergistically effective amount.

An interferon and/or an inhibitor of the MAPK signaling pathway may be present in the pharmaceutical composition in an amount ranging from about 0.005% (w/w) to about 100% (w/w), from about 0.01% (w/w) to about 90% (w/w), from about 0.1% (w/w) to about 80% (w/w), from about 1% (w/w) to about 70% (w/w), from about 10% (w/w) to about 60% (w/w), from about 0.01% (w/w) to about 15% (w/w), or from about 0.1% (w/w) to about 20% (w/w).

An interferon and an inhibitor of the MAPK signaling pathway may be present in two separate pharmaceutical compositions to be used in a combination therapy.

The present agents or pharmaceutical compositions may be administered by any route, including, without limitation, oral, transdermal, ocular, intraperitoneal, intravenous, ICV, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The present composition may be administered parenterally or systemically.

The pharmaceutical compositions of the present invention can be, e.g., in a solid, semi-solid, or liquid formulation. Intranasal formulation can be delivered as a spray or in a drop; inhalation formulation can be delivered using a nebulizer or similar device; topical formulation may be in the form of gel, ointment, paste, lotion, cream, poultice, cataplasm, plaster, dermal patch aerosol, etc.; transdermal formulation may be administered via a transdermal patch or iontorphoresis. Compositions can also take the form of tablets, pills, capsules, semisolids, powders, sustained release formulations, solutions, emulsions, suspensions, elixirs, aerosols, chewing bars or any other appropriate compositions.

The composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

To prepare such pharmaceutical compositions, one or more of compound of the present invention may be mixed with a pharmaceutical acceptable excipient, e.g., a carrier, adjuvant and/or diluent, according to conventional pharmaceutical compounding techniques.

Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, preservatives and adjuvants, see Remington's Pharmaceutical Sciences, edited by E. W. Martin (Mack Publishing Company, 18th ed., 1990). Additional excipients, for example sweetening, flavoring and coloring agents, may also be present.

The pharmaceutically acceptable excipient may be selected from the group consisting of fillers, e.g. sugars and/or sugar alcohols, e.g. lactose, sorbitol, mannitol, maltodextrin, etc.; surfactants, e.g. sodium lauryle sulfate, Brij 96 or Tween 80; disintegrants, e.g. sodium starch glycolate, maize starch or derivatives thereof; binder, e.g. povidone, crosspovidone, polyvinylalcohols, hydroxypropylmethylcellulose; lubricants, e.g. stearic acid or its salts; flowability enhancers, e.g. silicium dioxide; sweeteners, e.g. aspartame; and/or colorants. Pharmaceutically acceptable carriers include any and all clinically useful solvents, dispersion media, coatings, antibacterial and antifungal agents, isotonic and absorption delaying agents and the like.

The pharmaceutical composition may contain excipients for modifying, maintaining or preserving, for example, the pH, osmolarity, viscosity, clarity, color, isotonicity, odor, sterility, stability, rate of dissolution or release, adsorption or penetration of the composition. Suitable excipients include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA), ethylene glycol tetraacetic acid (EGTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

Oral dosage forms may be tablets, capsules, bars, sachets, granules, syrups and aqueous or oily suspensions. Tablets may be formed form a mixture of the active compounds with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound, may be prepared by known methods. The contents of the capsule may be formulated using known methods so as to give sustained release of the active compounds. Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient or they may be added to a suitable liquid carrier (e.g. water) before ingestion. The granules may contain disintegrants, e.g. an effervescent pair formed from an acid and a carbonate or bicarbonate salt to facilitate dispersion in the liquid medium. U.S. Pat. No. 8,263,662.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Additional compositions include formulations in sustained or controlled delivery, such as using liposome or micelle carriers, bioerodible microparticles or porous beads and depot injections.

The present compound(s) or composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, inhalation, or combinations of these methods. For example, one mode of administration that can be used involves continuous intravenous infusion. The infusion of the compositions of the invention can, if desired, be preceded by a bolus injection.

The amount of interferon (e.g., a first amount) or the amount of the inhibitor of the mitogen-activated protein kinase (MAPK) signaling pathway (e.g., a second amount) that may be used in the combination therapy may be a therapeutically effective amount, a sub-therapeutically effective amount or a synergistically effective amount. The amounts are dosages that achieve the desired synergism.

As used herein, the term “therapeutically effective amount” is an amount sufficient to treat a specified disorder or disease or alternatively to obtain a pharmacological response treating a disorder or disease.

Methods of determining the most effective means and dosage of administration can vary with the composition used for therapy, the purpose of the therapy, the target cell being treated, and the subject being treated. Single or multiple administrations can be carried out with the dose level and pattern being selected by the treating physician. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

For example, the interferon or the inhibitor of the MAPK pathway may be administered at about 0.0001 mg/kg to about 500 mg/kg, about 0.01 mg/kg to about 200 mg/kg, about 0.01 mg/kg to about 0.1 mg/kg, about 0.1 mg/kg to about 100 mg/kg, about 10 mg/kg to about 200 mg/kg, about 10 mg/kg to about 20 mg/kg, about 5 mg/kg to about 15 mg/kg, about 0.0001 mg/kg to about 0.001 mg/kg, about 0.001 mg/kg to about 0.01 mg/kg, about 0.01 mg/kg to about 0.1 mg/kg, about 0.1 mg/kg to about 0.5 mg/kg, about 0.5 mg/kg to about 1 mg/kg, about 1 mg/kg to about 2.5 mg/kg, about 2.5 mg/kg to about 10 mg/kg, about 10 mg/kg to about 50 mg/kg, about 50 mg/kg to about 100 mg/kg, about 100 mg/kg to about 250 mg/kg, about 0.1 μg/kg to about 800 μg/kg, about 0.5 μg/kg to about 500 μg/kg, about 1 μg/kg to about 20 μg/kg, about 1 μg/kg to about 10 μg/kg, about 10 μg/kg to about 20 μg/kg, about 20 μg/kg to about 40 μg/kg, about 40 μg/kg to about 60 μg/kg, about 60 μg/kg to about 100 μg/kg, about 100 μg/kg to about 200 μg/kg, about 200 μg/kg to about 300 μg/kg, or about 400 μg/kg to about 600 μg/kg. In some embodiments, the dose is within the range of about 250 mg/kg to about 500 mg/kg, about 0.5 mg/kg to about 50 mg/kg, or any other suitable amounts.

The effective amount of the interferon or the inhibitor of the MAPK pathway for the combination therapy may be less than, equal to, or greater than when the agent is used alone.

The amount or dose of an inhibitor of any component of the MAPK pathway may range from about 0.01 mg to about 10 g, from about 0.1 mg to about 9 g, from about 1 mg to about 8 g, from about 1 mg to about 7 g, from about 5 mg to about 6 g, from about 10 mg to about 5 g, from about 20 mg to about 1 g, from about 50 mg to about 800 mg, from about 100 mg to about 500 mg, from about 600 mg to about 800 mg, from about 800 mg to about 1 g, from about 0.01 mg to about 10 g, from about 0.05 μg to about 1.5 mg, from about 10 μg to about 1 mg protein, from about 0.1 mg to about 10 mg, from about 2 mg to about 5 mg, from about 1 mg to about 20 mg, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg.

The dose of an interferon may range from about 0.1 μg/day to about 1 mg/day, from about 10 μg/day to about 200 μg/day, from about 20 μg/day to about 150 μg/day, from about 0.1 μg/day to about 125 μg/day, from about 1 μg/day to about 20 μg/day, or about 4.5 μg/day to about 30 μg/day.

The dose of an interferon may also range from about 1 million international units (MU) to about 800 MU, from about 1 MU to about 10 MU, from about 20 MU to about 40 MU, from about 2 MU to about 15 MU, from about 5 MU to about 25 MU, from about 50 MU to about 100 MU, from about 150 MU to about 250 MU, from about 300 MU to about 400 MU, from about 500 MU to about 600 MU, or other doses.

Different dosage regimens may be used. In some embodiments, a daily dosage, such as any of the exemplary dosages described above, is administered once, twice, three times, or four times a day for at least three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

### Kits

The present invention also provides for a kit for use in the treatment or prevention of cancer or other conditions. Kits according to the invention include package(s) (e.g., vessels) comprising agents or compositions of the invention. The kit may include (i) an interferon, and (ii) an inhibitor of the MAPK signaling pathway. The interferon and the inhibitor of the MAPK signaling pathway may be present in the pharmaceutical compositions as described herein. The interferon and the inhibitor of the MAPK signaling pathway may be present in unit dosage forms.

Examples of pharmaceutical packaging materials include, but are not limited to, bottles, tubes, inhalers, pumps, bags, vials, containers, syringes, bottles, and any packaging material suitable for a selected formulation and intended mode of administration and treatment.

Kits can contain instructions for administering agents or compositions of the invention to a patient. Kits also can comprise instructions for uses of the present agents or compositions. Kits also can contain labeling or product inserts for the inventive compounds. The kits also can include buffers for preparing solutions for conducting the methods. The instruction of the kits may state that the combination of the interferon and the inhibitor of the MAPK pathway produces a synergistic effect on the cancer compared to the effect of the interferon alone or the effect of the inhibitor of the MAPK pathway alone.

Subjects, which may be treated according to the present invention include all animals which may benefit from administration of the agents of the present invention. Such subjects include mammals, preferably humans, but can also be an animal such as dogs and cats, farm animals such as cows, pigs, sheep, horses, goats and the like, and laboratory animals (e.g., rats, mice, guinea pigs, and the like).

The following are examples of the present invention and are not to be construed as limiting.

### EXAMPLES

### Summary

In order to explain the phenotypic variability in response to MAPK inhibition, we studied the transcriptional response to this inhibition. While most studies had used correlation between genetic and genomic features and phenotypic outcome to identify predictive features (Barretina et al., 2012; Garnett et al., 2012), we took a different approach. We used pre- and post-MEK inhibition expression data in a panel of genetically diverse cell lines to better understand the targets and pathways regulated by ERK-MAPK. We then used these regulation patterns, and how they differed between tumors, to explain the variability in response to treatment. In this study, genes with changes in their mRNA levels following MEK inhibition were defined as targets of the MAPK pathway.

We found extensive heterogeneity in the transcriptional response to MEK inhibition between cell lines. Although all cell lines harbor a MAPK pathway activating mutation (either NRAS or BRAF), a vast majority of MAPK targets are context-specific—under the control of the pathway in only a subset of cell lines (as used herein, the term “context” refers to any subset of the cell lines, with or without a known, shared and unique genetic feature). As these differences could reveal the molecular mechanisms underlying phenotypic variance, we developed a computational tool, COSPER (COntext SPEcific Regulation), to identify context-specific targets using pre- and post-perturbation gene expression data.

Analysis with COSPER revealed that the IFN-Type I pathway presents context-specific behavior. While studying this pathway, we found a strong cytotoxic synergy between two unrelated therapies for melanoma—Type-I Interferon (IFN/β) and MEK inhibitor. We show that cell lines with high basal activity of the interferon pathways are resistant to MEK inhibition alone or its combination with IFNα/β. We identified a genetic lesion, deletion of the interferon locus, which leads to differential basal activity level of the interferon pathway and predicts the cytotoxic response of MEK inhibition.

We have now studied the effects of MEK inhibition on the transcriptome in a panel of melanoma cell lines and found that most targets are context-specific—under the influence of the pathway in only a subset of cell lines. We developed a computational method to identify context-specific targets of MAPK, and found differences in the activity levels and regulation of the interferon pathway. Examination of the pathway and its interaction with MAPK revealed a strong synergy in the cytotoxic effects of IFNα/β treatment and MEK inhibition. Taken together, our results suggest that the interferon pathway plays an important role, and predicts, the response to MAPK inhibition in melanoma. Our analysis demonstrates the value of system-wide perturbation data in predicting drug response.

### Example 1

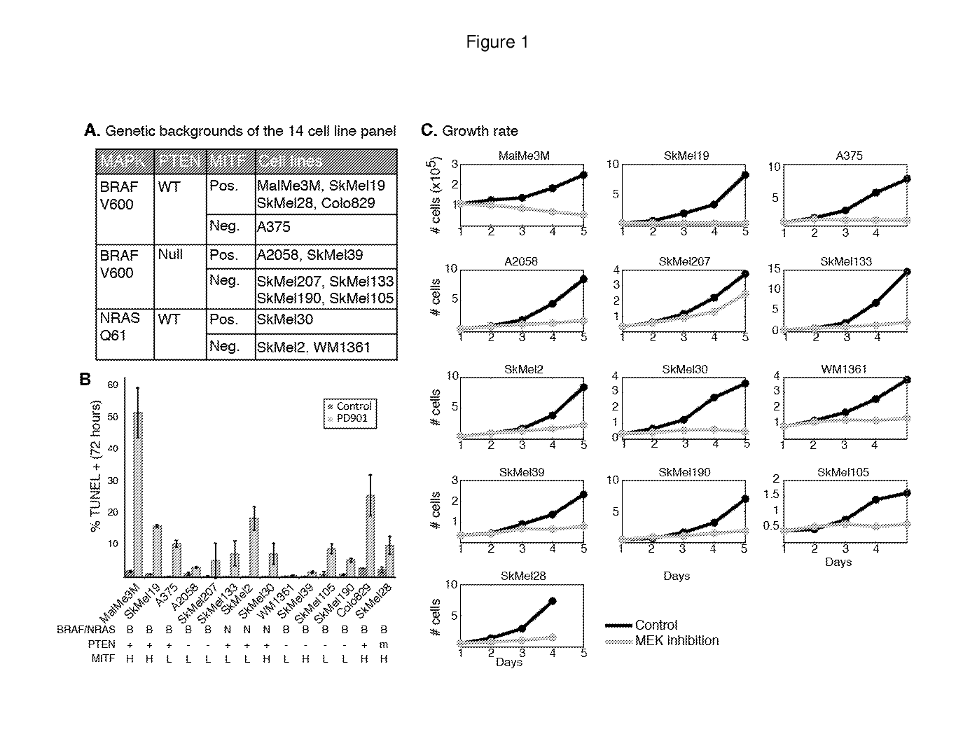

Cell lines harboring MAPK-activating mutations vary in their response to inhibition of the pathway, both in rate of proliferation and death (Xing et al., 2012). To characterize the targets and crosstalk of the ERK-MAPK pathway, we chose a panel of 14 genetically diverse melanoma cell lines. This panel represents the spectrum of common genetic aberrations in melanoma—MAPK mutations, MITF amplification and PTEN deletion (FIG. 1A).

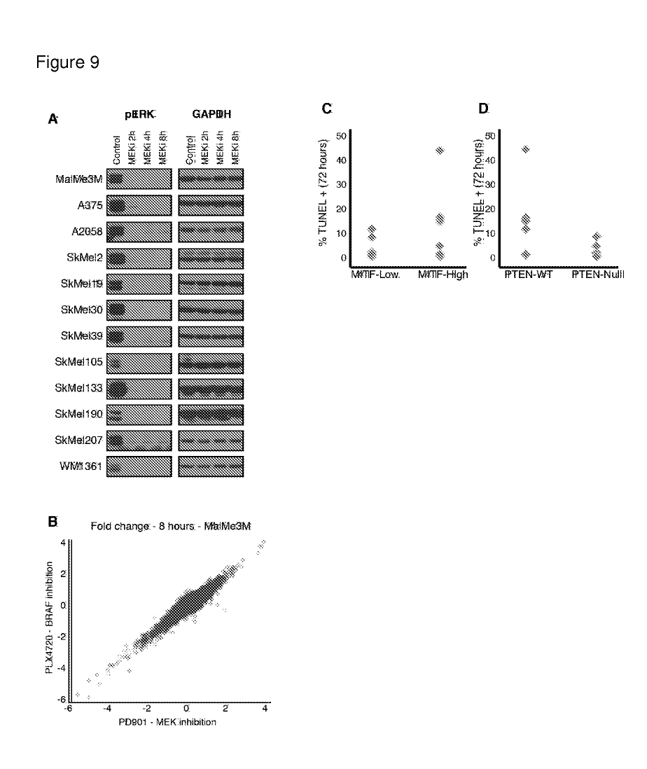

To compare the transcriptional and phenotypic response to MAPK pathway inhibition of both NRAS-mut and BRAF-mut cell lines we used a MEK inhibitor (PD325901, 50 nM) that fully inhibits the pathway in all cell lines at 8 hours (FIG. 9A), and not the clinically used BRAF inhibitor, which works on BRAF-mut cells only. A comparison of the MEK inhibitor with a BRAF inhibitor (PLX4720 (Tsai et al., 2008)) in a BRAF-V600E cell line shows almost identical transcriptional response, both in the genes affected and the extent of transcriptional change (FIG. 9B).

We first characterized the cell lines' phenotypic responses to MEK inhibition, in each of the 14 cell lines included in our panel. The cell lines display a wide range of cytotoxic responses, as well as differences in proliferation under MEK inhibition (FIGS. 1B and 1C). Notably, and contrary to previously published results (Barretina et al., 2012; Xing et al., 2012), we found that key genetic aberrations common in melanoma, including MITE and PTEN status, and MAPK mutation type, fail to fully explain the response heterogeneity (FIGS. 1B, 9C-9D).

### Heterogeneity in Transcriptional Response to MAPK Inhibition

To identify MAPK transcriptional targets, and how these differ across cell lines, we characterized the transcriptional response before and after MEK inhibition. We measured gene expression 8 hours following MEK inhibition to capture the peak of the transcriptional changes following inhibition (Pratilas et al., 2009).

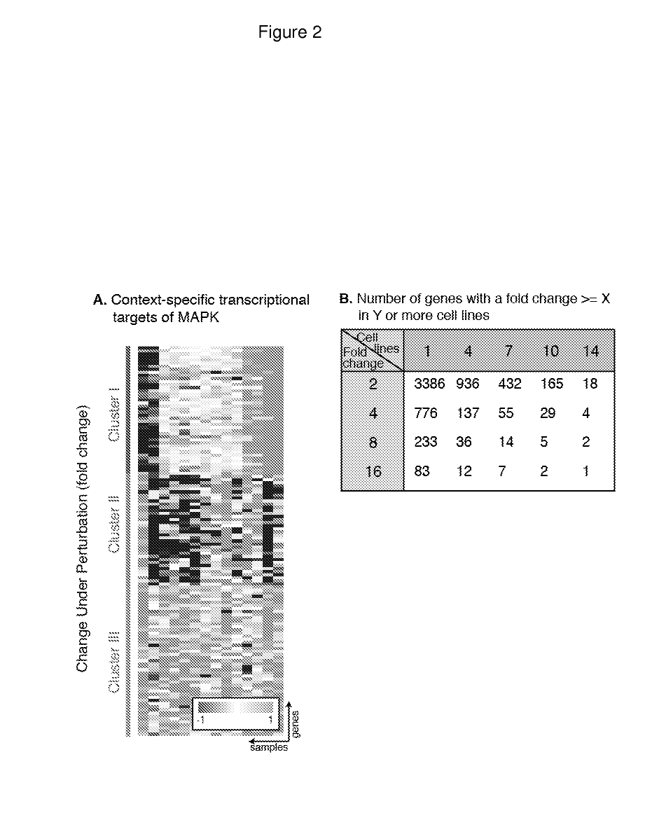

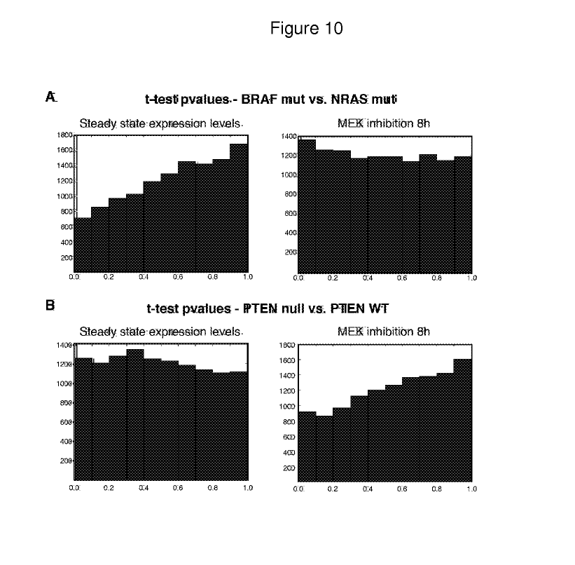

The most striking phenomenon observed in post inhibition data is the heterogeneity in response to MEK inhibition across different cell lines. Although all cell lines harbor a MAPK activating mutation, most downstream genes are regulated by the MAPK pathway in only a subset of the cell lines, and no two cell lines behave similarly (FIG. 2A). For example, only 18 genes change by >2 fold in all 14 cell lines, but 936 genes pass this threshold in 4 or more cell lines (FIG. 2B). Those context-specific targets are under the control of the MAPK pathway in only a subset of cell lines. The term “context” is used to represent a known or unknown genetic or genomic background that is shared by a subset of cell lines, but not by the others. Notably, we did not find a significant enrichment of genes regulated by MAPK only in BRAF-mut or NRAS-mut cell lines (FIG. 10A-B).

Our data show that MEK inhibition leads to different phenotypic responses in different cell lines, and that MAPK regulates different genes, and presumably different pathways, in different cell lines. We hypothesized that differential regulation of pathways and genes underlies the phenotypic variability, and identifying context-specific targets might explain it. Therefore, we investigated the patterns of context-specific regulation.

### Context-Specific Regulation

The first step in the analysis was to identify targets of the MAPK pathway using post-inhibition changes in expression levels. However, due to the heterogeneity of response, methods that classify genes as targets and non-targets by using fold-change thresholds are not suitable to identify MAPK targets. The numbers in FIG. 2B show that choosing an arbitrary fold-change threshold and number of tumors misclassifies genes. We therefore developed a new method that specifically searches for context-specific MAPK regulated genes using both pre- and post-perturbation data.

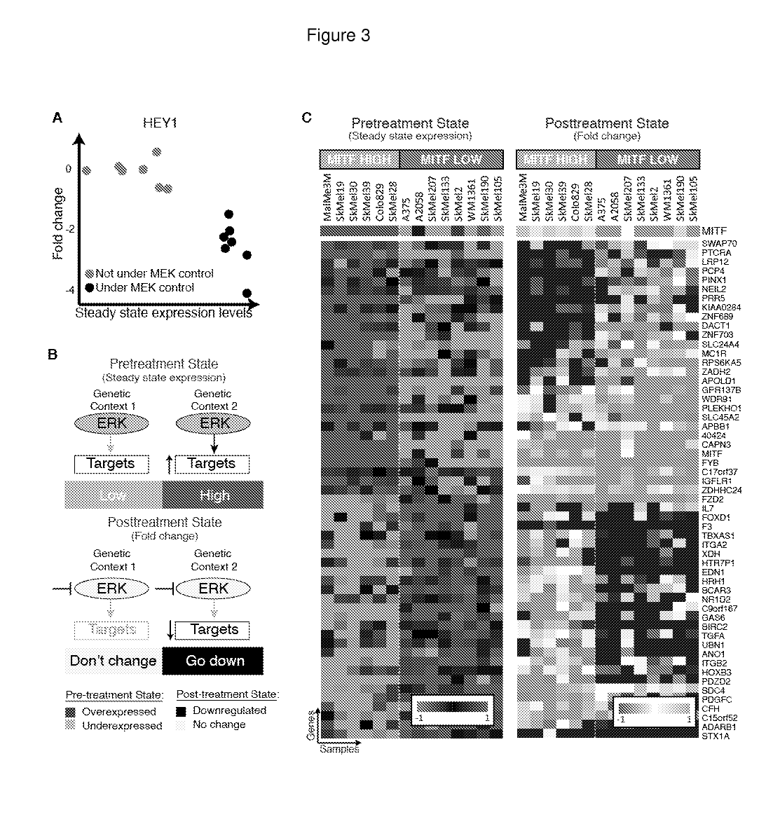

Some genes show distinct patterns of context-specific regulation both before and after MAPK inhibition. HEY1 is used as an example of a context-specific regulated gene (FIG. 3A). HEY1 has two states, or contexts, that are detectable both in pre- and post-inhibition expression levels. In one context (i.e. one set of cell lines) it is not under the control of MAPK, and shows low basal expression levels when MAPK is active, and its expression doesn't change after inhibition of the pathway (FIG. 3A). In the second group of cell lines, HEY1 is up-regulated by MAPK and therefore shows high basal expression levels before pathway inhibition, and its expression drops following MEK inhibition.

As genes are often co-regulated, we expect clusters of context-specific co-regulated genes (FIG. 3B). While gene expression data are noisy, using clusters of genes to identify contexts and context-specific targets enables us to computationally reduce the experimental noise. Moreover, we increase the probability that the association between a context and a gene is a product of an underlying biological phenomenon rather than a spurious association.

We developed a computational method—COSPER (COntext-SPEcific Regulation)—that uses pre- and post-inhibition transcriptional data to identify context-specific co-regulated clusters of genes.

### COSPER Identifies Context-Specific MAPK Regulated Genes

COSPER can be viewed as a bi-clustering algorithm—designed to identify gene clusters that show context-specific regulation patterns (FIG. 3B). In each cluster, the cell lines are divided into two groups, or contexts, and the genes have a distinct but different behavior in each context, both before and after pathway inhibition. As demonstrated in the case of HEY1, combining data from both pre- and post-pathway inhibition focuses the search to genes that are likely to be regulated by the MAPK pathway. By identifying the genes regulated by the pathway in only a subset of cell lines, e.g. sensitive versus resistant, COSPER helps focus the analysis on genes and pathways that are likely to contribute to the phenotypic response to pathway inhibition.

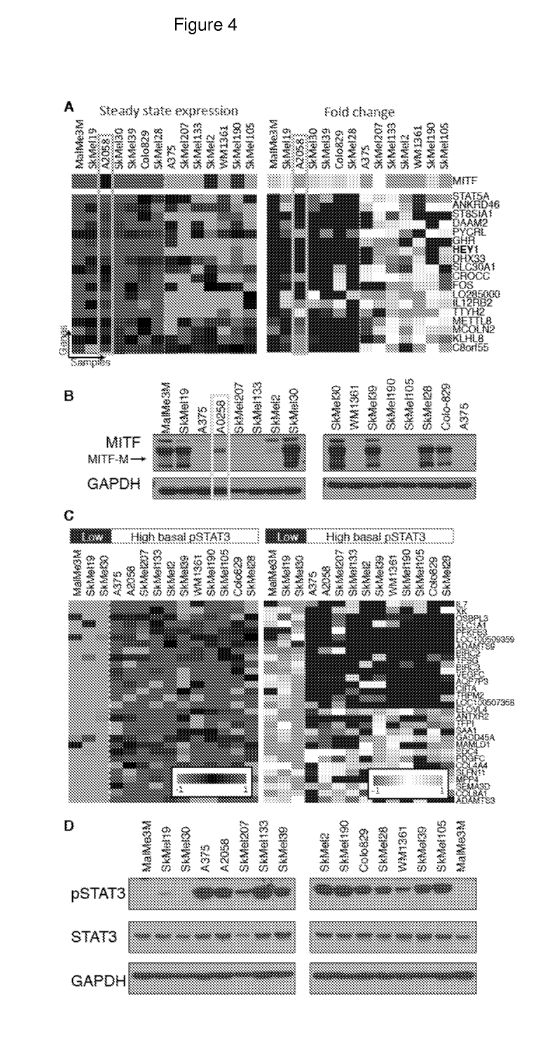

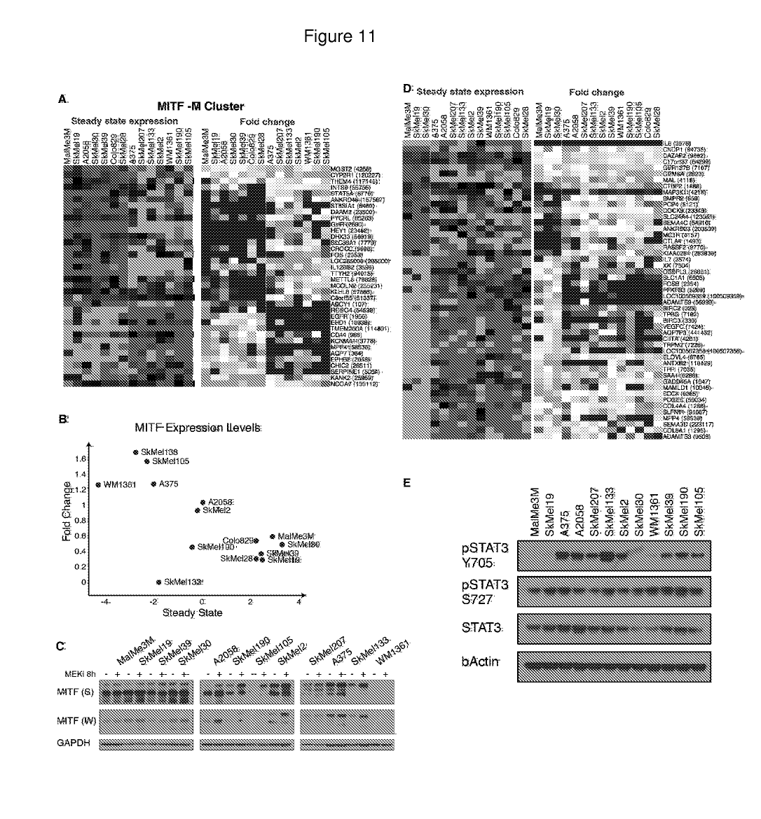

COSPER is not restricted to the patterns depicted in FIG. 3A, and can identify any context-specific pattern of regulation (FIG. 3C). Overall, COSPER identified 70 context-specific clusters with 5 genes or more, and assigned a total of 1024 genes to clusters (genes are allowed to belong to more than one cluster, list of all clusters appears in Table S1). Fifteen clusters associate with MITF, containing 401 genes in total. These clusters either have a perfect correlation with MITF expression, such as the cluster in FIG. 3C, or have 1-2 cell lines that “switch sides”—they behave similarly to cell lines with the opposite MITF status (FIGS. 4A and 11A, which include HEY1).

Notably, none of the clusters correlate with the oncogenic activation of MAPK (BRAF or NRAS), or with the cells' PTEN status. Moreover, we also explicitly tested for genes correlated with these aberrations, but no gene's expression was found to be significantly associated with these mutations (FIG. 10).

### Inferring Pathway Activity Using COSPER

COSPER identifies clusters of genes downstream of MAPK that show context-specific behavior. Using standard gene set enrichment analysis methods, we can postulate the pathways that govern the differential expression of those genes, and the activity of the clusters' regulators.

For example, the clusters in FIGS. 3C and 4A demonstrate the different roles of MITF isoforms. While the cluster in FIG. 3C correlates with MITF mRNA expression, the cluster in FIG. 4A correlates with the abundance of the MITF-M protein isoform (FIG. 4B). MITF itself is also regulated by MAPK, both at the mRNA and protein levels (FIGS. 11B, 11C), which explains the regulation of MITF targets by the MAPK pathway.

The different functional annotations of the genes in the two clusters suggest that different MITF isoforms regulate different processes. The promoters for genes in the MITF-M cluster are highly enriched for the MITF binding site (CACATG) (Levy et al., 2006) (p-value=10⁻³ compared with 0.7 for genes in MITF-expression cluster). However, the MITF-expression cluster, but not the MITF-M cluster, is enriched for the GO annotation “melanocyte differentiation” (q-value=10⁻⁴), suggesting that another isoform of MITF is responsible for cellular differentiation.

An additional cluster COSPER identified is enriched for the STAT3 pathway (FIGS. 4C, 11D). Gene ontology enrichment analysis found that the genes in the cluster are enriched for cytokine-cytokine receptor pathway (q-value<10⁻³), and with miR-19 and miR-17 (q-value<10⁻³), two miRs known to be regulated by pSTAT3 (Dai et al., 2011; Zhang et al., 2012), which led us to suspect that this cluster is associated with STAT3 regulation. We confirmed these predictions by measuring STAT3 activity in the cell lines. Levels of pSTAT3-Y705, an indicator for STAT3 activity, but not of pSTAT3-S727, match the cluster's contexts (FIG. 4D, 11E).

Using the MITF and STAT3 examples, we showed that COSPER infers both network state and interactions between pathways. However, when running COSPER on steady-state data alone, the resulting clusters are much larger, less specific, and therefore less informative than the clusters resulting from using both conditions (see comparison analysis in supplementary information). Moreover, post-inhibition data enable the identification of genes regulated by the MAPK pathway. Therefore, post-inhibition mRNA expression data play a critical role in identifying the state and interconnectivity of pathways.

### Interferon-STAT1 Pathway is Differentially Regulated in Cell Lines

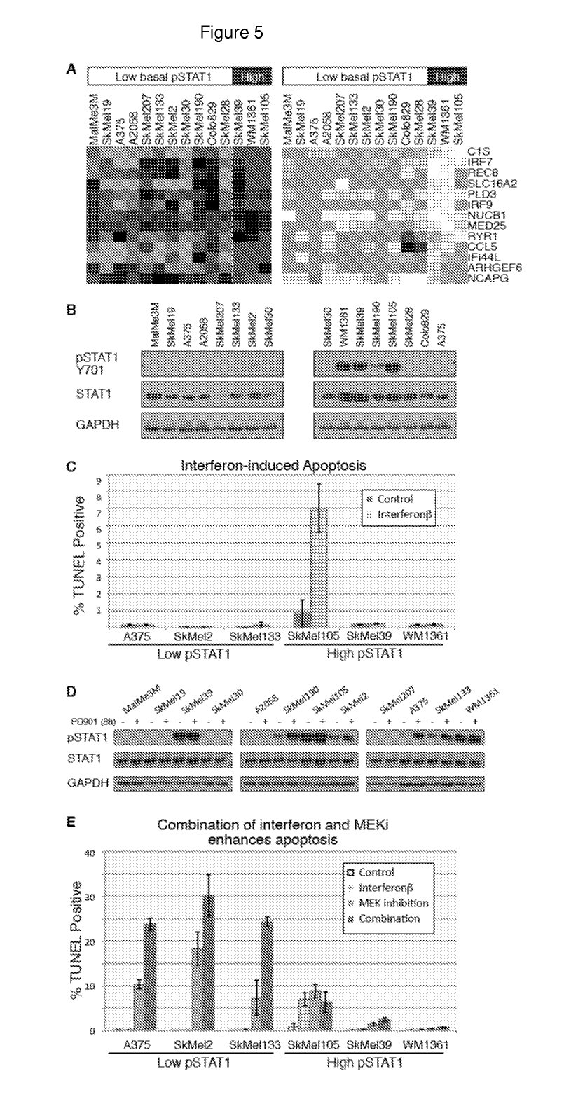

COSPER also identified a cluster that contains several interferon targets, IRF7, IRF9, CCL5 and IFI44L (FIG. 5A), which reflect the activity of the Type I interferon pathway (Hecker et al., 2013). Since Type I interferon (IFNα/β) is one of the few approved drugs for metastatic melanoma, we decided to focus on this cluster.

The cluster splits the cell lines into two groups; the first contains 3 cell lines with an up-regulation of interferon response genes, while cell lines in the second context express these genes at lower levels. Levels of pSTAT1-Y701, an indicator of the interferon-STAT1 activity levels (Platanias, 2005), confirmed that the high basal expression levels of the pathway targets correspond with high signaling activity of the pathway (FIG. 5B). Notably, the cell lines with up-regulation of the STAT1-interferon response genes are not the same 3 cell lines with low activity of STAT3.

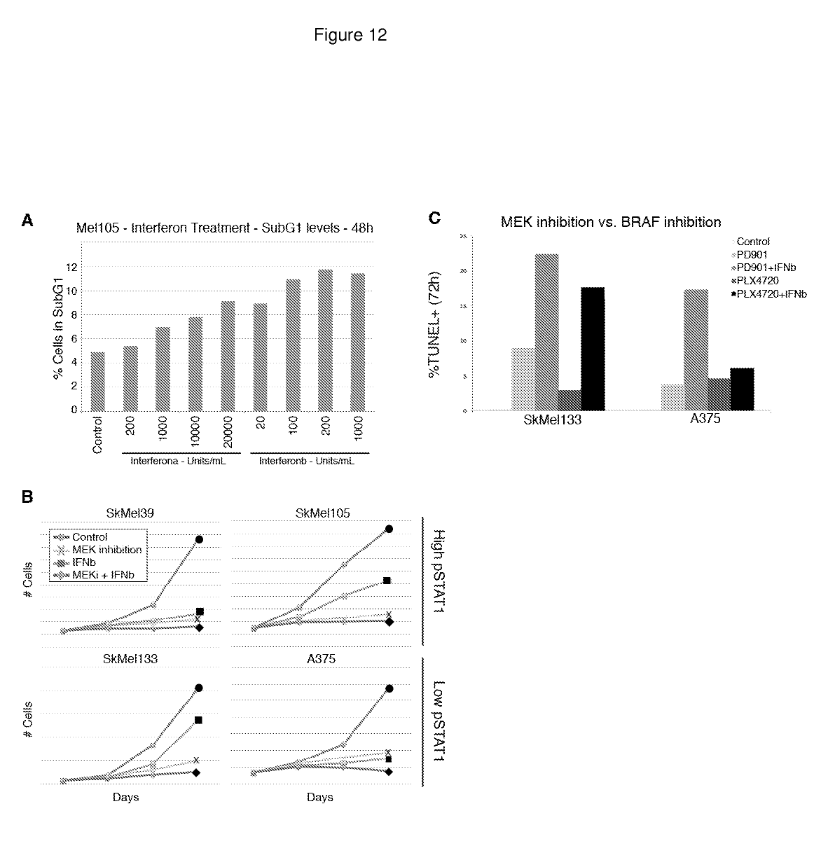

High basal activity of the STAT1-interferon pathway has been previously shown to be necessary, but not sufficient, for IFNα/β-induced apoptosis (Jackson et al., 2003). To test this claim, 3 low- and 3 high-pSTAT1 cell lines were treated with IFNβ and apoptosis levels were assessed by TUNEL (terminal deoxynucleotidyl transferase dUTP nick end labeling) assay. All low-pSTAT1 and 2 high-pSTAT1 cell lines were resistant to the cytotoxic effects of IFNβ, and one high-pSTAT1 cell line was marginally sensitive (FIG. 5C). Both IFNα and IFNβ were tested, and as previously shown (Leaman et al., 2003), IFNβ led to a stronger apoptotic response than IFNα (FIG. 12A); thus, IFNβ was chosen for further analysis. Our results confirmed the previous findings that STAT1 activity is necessary, but not sufficient, for IFNα/β sensitivity.

IFNβ Synergizes with MEK Inhibition to Increase Apoptosis in Low pSTAT1 Cell Lines

According to the expression data, MEK inhibition leads to an up-regulation of the IFNα/β pathway. Analysis of protein levels by Western blots indicates an increase in pSTAT1 levels after MEK inhibition, confirming a crosstalk between MAPK and STAT1 (FIG. 5D).

The cytotoxic effect of MEK inhibition on both high- and low-pSTAT1 cell lines was assessed. We found that high-pSTAT1 cell lines are mostly resistant to the cytotoxic effects of MEK inhibition, while low-pSTAT1 cells are sensitive (FIG. 5E). Notably, both groups contain NRAS and BRAF mutant cell lines, and cell lines with high and low MITF expression, although both MITF-low cell lines and NRAS mutant cell lines have been previously reported to be less sensitive to MAPK pathway inhibition (Barretina et al., 2012; Solit et al., 2006). Moreover, the results show that the cytotoxic response of MEK inhibition is independent of its cytostatic response. For example, SkMel133 continues to grow rapidly under MEK inhibition (FIG. 1C), but has relatively high apoptosis levels under MEK inhibition.

We then examined the cytotoxic effect of the combination of MEK inhibition and IFNβ. While IFNβ as a single agent has no cytotoxic effect on low-pSTAT1 cell lines, it notably enhances the cytotoxic response of MEK inhibition, increasing TUNEL-positive cells by almost two-fold (FIG. 5E, 12B). Moreover, while low-pSTAT1 cell lines show a strong sensitivity to the combination of MEK inhibition and IFNβ, high pSTAT1 cell lines seem to be resistant to the cytotoxic effects of both MEK inhibition alone and the dual treatment (FIG. 5E). To confirm that the synergy between MAPK pathway inhibition and IFNβ is not specific to MEK inhibition, we show a similar synergy, albeit slightly weaker, between a BRAF inhibitor (PLX4720) and IFNβ (FIG. 12C).

### Transcriptional Response to IFN is Similar in all Cell Lines

Our data demonstrated that basal activation level of the interferon pathway predicts the cytotoxic response to MEK inhibition, and to its combination with IFNα/β. We hypothesized that these phenotypic difference are associated with changes in the interferon pathway and its response to IFNα/β treatment. We therefore characterized the signaling and transcriptional responses to IFNβ and MEK inhibition.

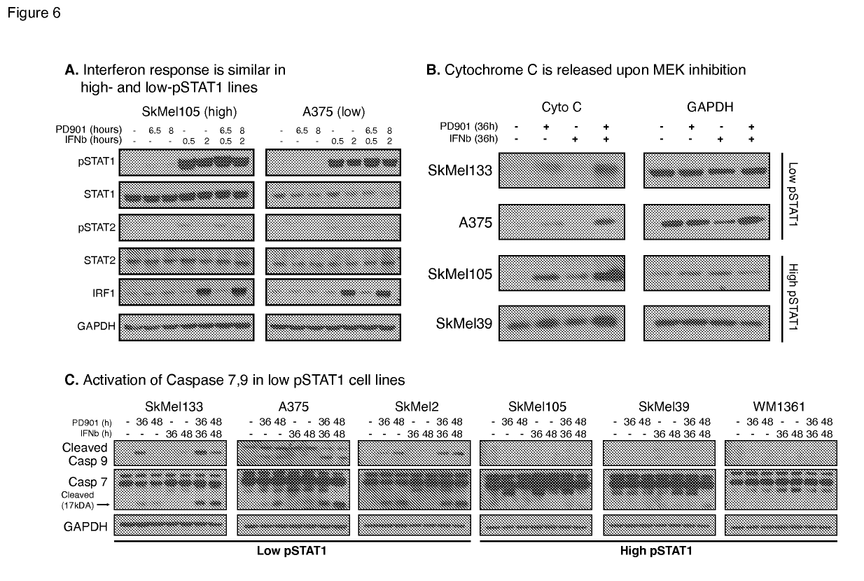

Western blots show that activation of STAT1 by IFNβ is identical, in both timing and extent, when comparing a low-pSTAT1 cell line to a high-pSTAT1 cell line (FIG. 6A, 13A). IFNβ treatment quickly elevates pSTAT1 levels and activates the interferon transcription program, as assessed by IRF1 and IRF7 levels, in both cell lines. Moreover, inhibition of MEK does not alter the timing or extent of the IFNβ response (FIG. 6A). Interestingly, we found that the levels of pSTAT1 in the so-called “high-pSTAT1 cell lines” are substantially lower than pSTAT1's levels following IFNβ treatment (FIG. 6A compared with FIG. 5B).

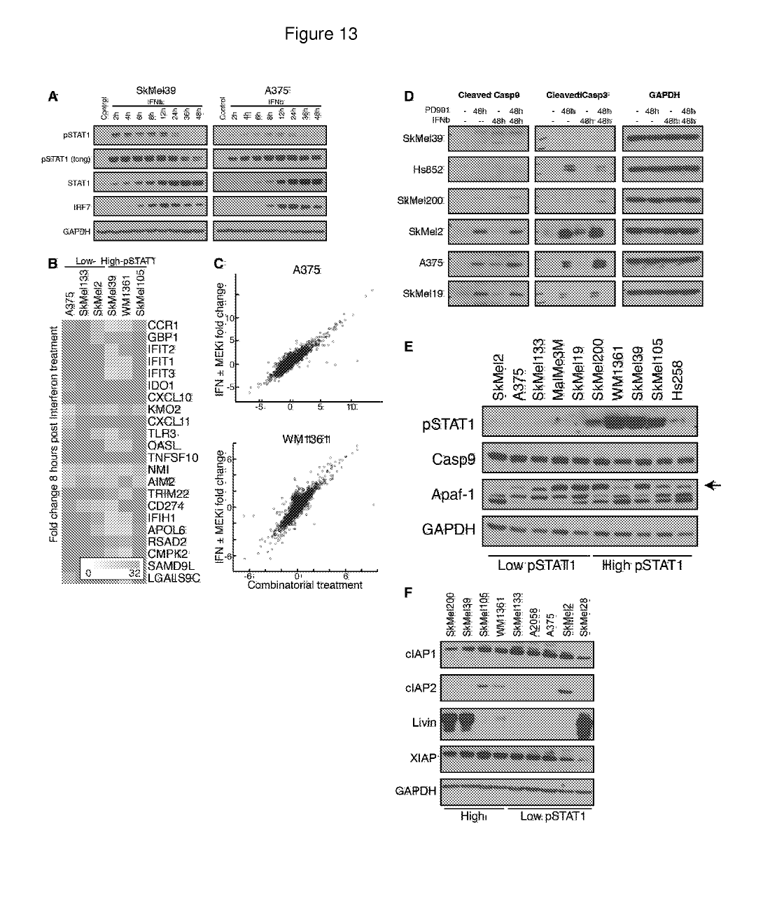

To search for more global regulatory differences in the interferon response, and to assess the effects of IFNβ more quantitatively, we measured gene expression levels 8 hours after treatment with PD325901, IFNβ or their combination in three low- and three high-pSTAT1 cell lines. All cell lines show a dramatic increase (up to 100 fold) in the expression of IFN targets following IFNβ treatment, confirming that the interferon response pathway is present and active in both contexts (FIG. 13B). Furthermore, no significant differences in the transcriptional response following IFNβ treatment between the low- and high-pSTAT1 cells is apparent after 8 hours of treatment. Additionally, MEK inhibition does not alter the IFNβ response, and does not synergize with IFNβ to induce transcription of any other genes (FIG. 13C).

These data suggest that the differences in the phenotypic response are not due to the basal activation level of the interferon pathway, as the transcriptional response to IFNβ is not different between high- and low-pSTAT1 cell lines.

The Caspase Pathway is Only Activated in Low pSTAT1 Cell Lines

Since the transcriptional response to IFNβ fails to explain the differences in the cytotoxic response between the cell lines, we moved to characterize the apoptotic pathway directly.

The intrinsic apoptotic pathway is initiated by the release of cytochrome C (CytoC) from the mitochondria, which together with Apaf-1, cleaves and activates initiator and executioner caspases (Bratton and Salvesen, 2010). Surprisingly, we found that inhibition of MEK is sufficient to induce release of CytoC in all cell lines, and the release is enhanced by co-treatment with IFNβ (FIG. 6B). However, although MEK inhibition initiates the intrinsic pathway in high-pSTAT1 cell lines, and this response is enhanced by IFN, these cell lines fail to undergo apoptosis.

CytoC release leads to apoptosis by activating the caspase pathway. We found that caspase 9, an initiator caspase, and caspases 7 and 3, executioner caspases, are cleaved following the release of CytoC by MEK inhibition in low-pSTAT1 cell lines only (FIGS. 6C and 13D). Combinatorial treatment leads to a stronger and faster activation of these two caspases, but IFNβ treatment alone does not activate them (FIGS. 6C, 13D). Importantly, caspases are not cleaved in high-pSTAT1 cell lines, although CytoC is released. This lack of activation may explain their cytotoxic resistance to treatment. To confirm the association between pSTAT1 levels and caspase activation, we extended our panel to 10 cell lines, adding 2 additional high- and 2 additional low-pSTAT1 cell lines. As with the original set of cell lines, caspases are cleaved only in low-pSTAT1 cell lines (FIG. 13D).

Additional components play part in the activation of the caspase pathway. APAF-1 forms the apoptosome with CytoC and activates the caspases (Soengas et al., 2001). Other proteins, such as cIAP1-2, XIAP and others, inhibit the pathway. We therefore assessed the levels of these proteins in our cell line panel, but found no correlation between their levels and the cytotoxic response to the treatments (FIG. 13E-F). Additionally, we confirmed that caspase 9, the upstream caspase of the caspase pathway (Riedl and Shi, 2004), is expressed in comparable levels in all cell lines (FIG. 13E). Another possibility for the lack of caspase activation, which we can't rule out based on our data, is that the levels of CytoC release in high pSTAT1 cell lines are not sufficient to activate the pathway, although we failed to notice differences in CytoC release between low- and high-pSTAT1 cell lines using Western blots.

Deletion of Interferon Locus Correlates with Cytotoxic Response

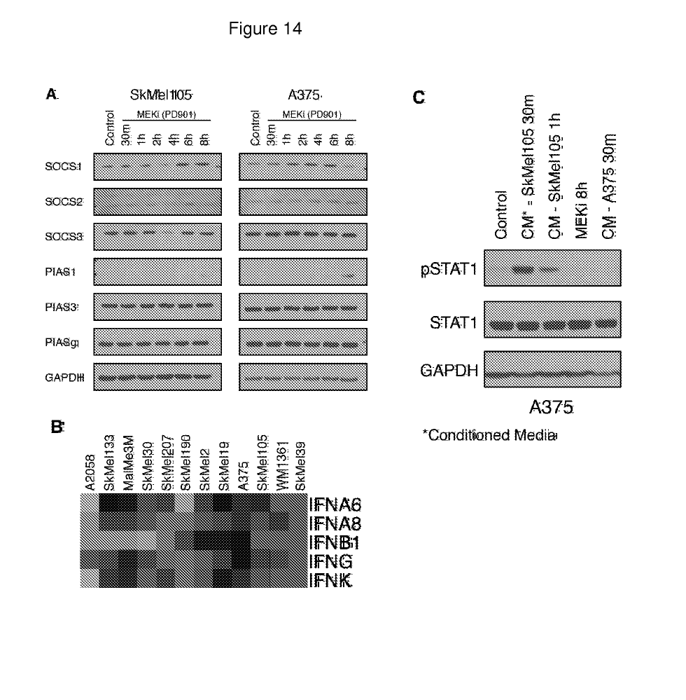

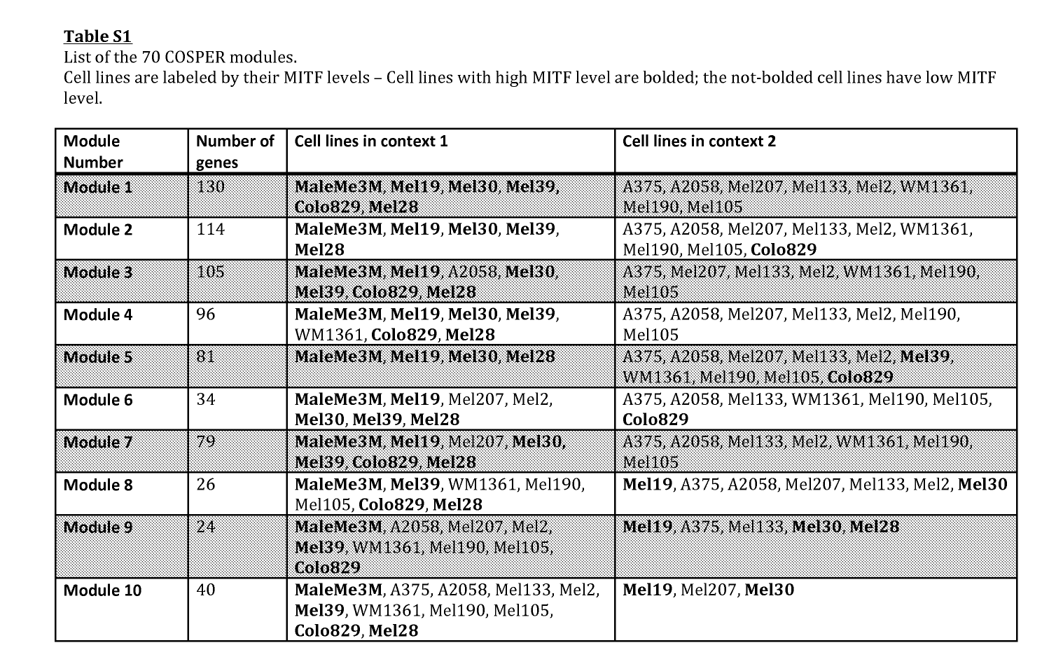

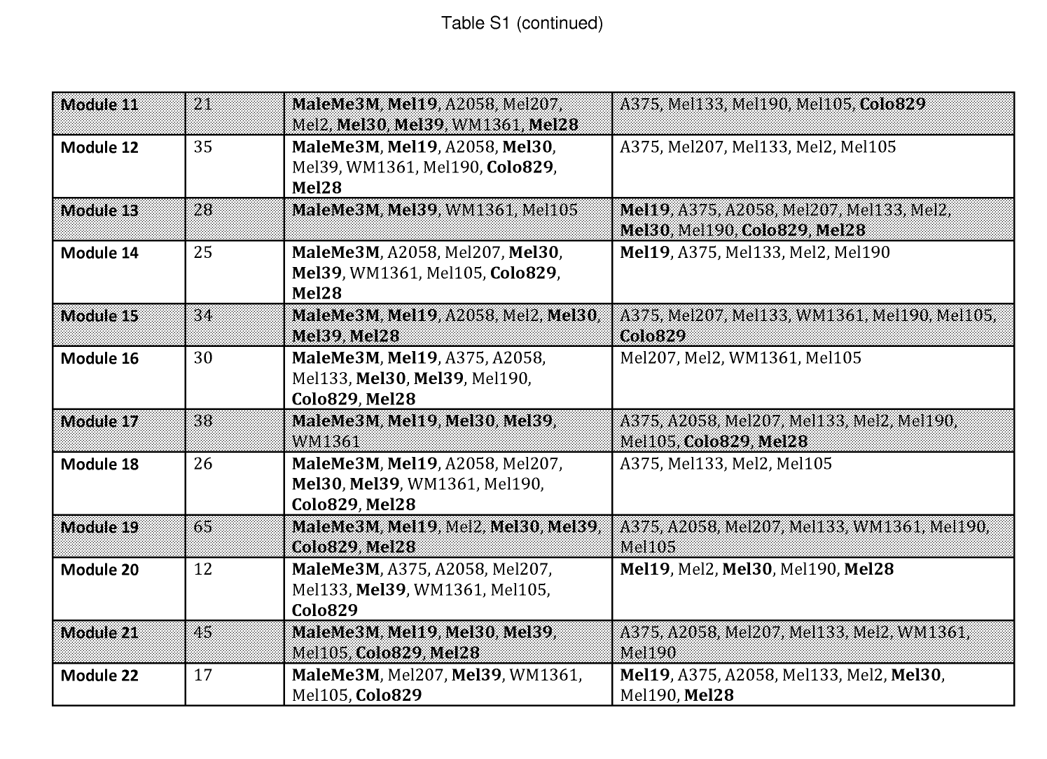

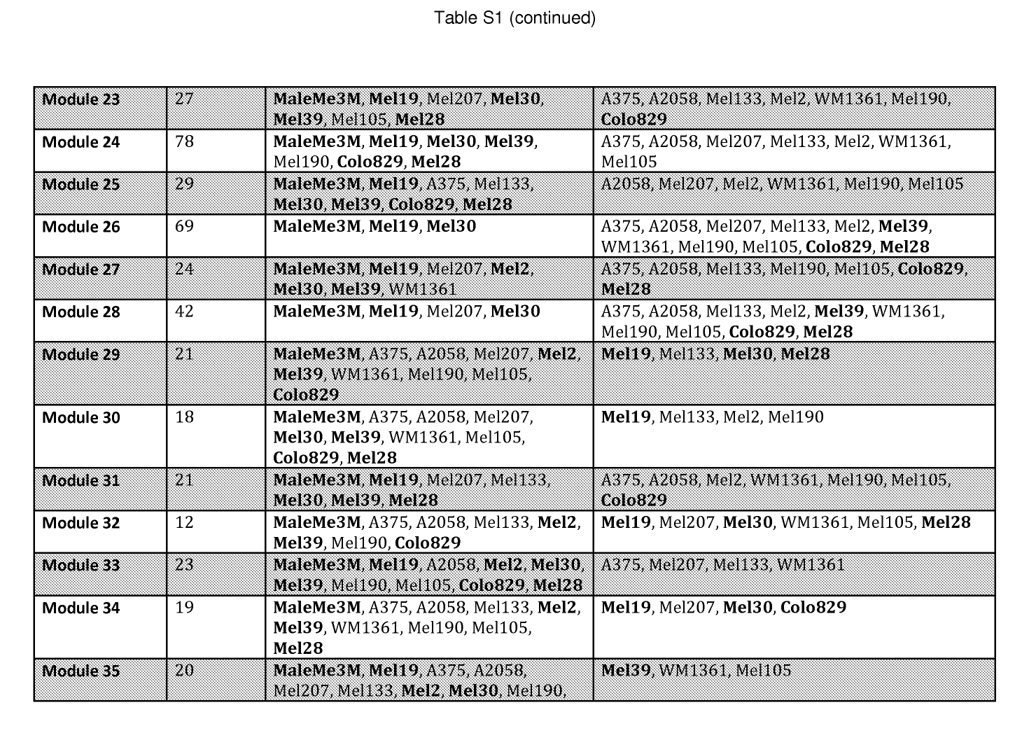

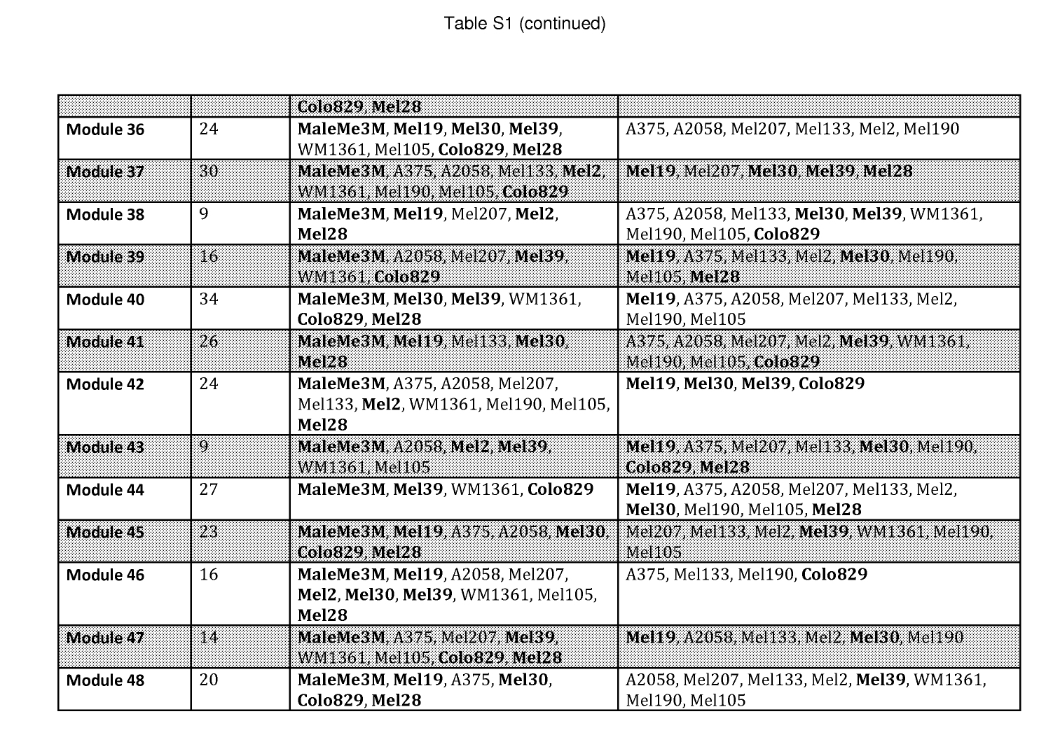

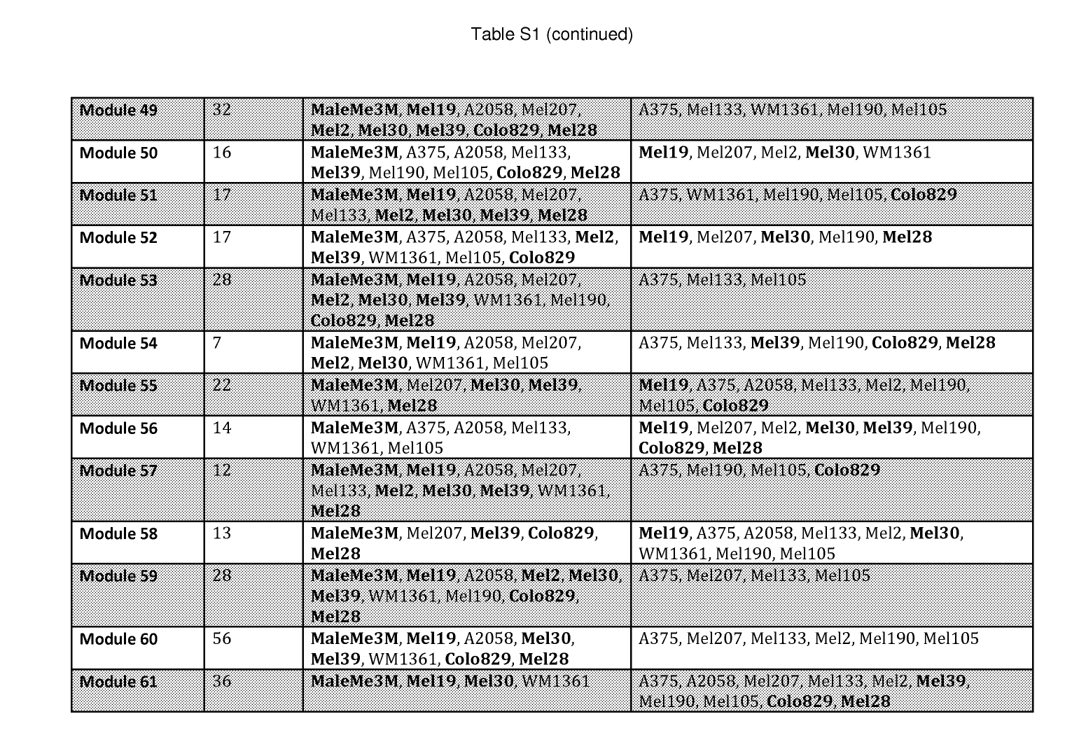

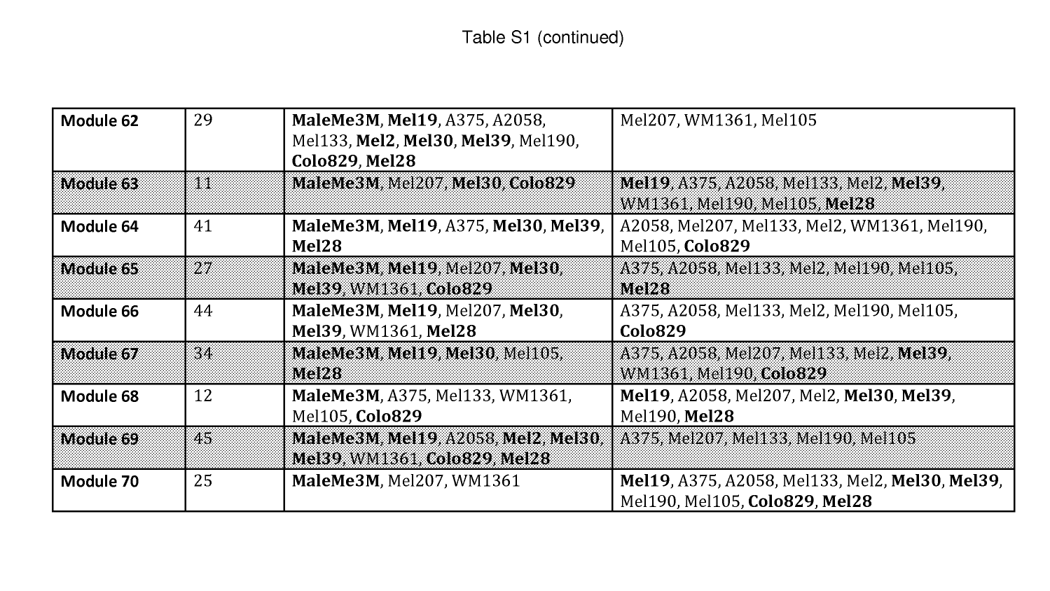

Basal activity of the interferon pathway predicts the cytotoxic response to MEK inhibition and its combination with IFNα/β. Levels of pathway inhibitors from the SOCS and PIAS family are similar in all cell lines and fail to explain the differences in the basal activation of the pathway (FIG. 14A). We therefore sought to identify genetic lesions that could be responsible for the differential basal activation of this pathway.

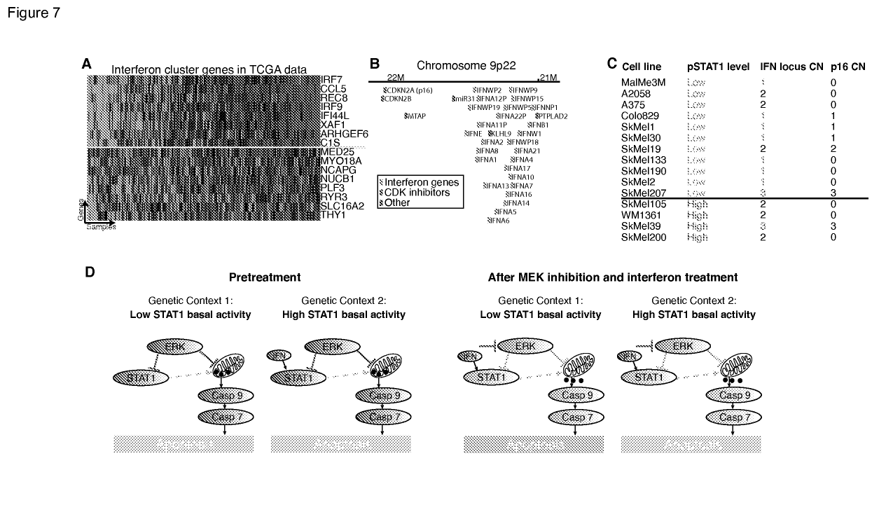

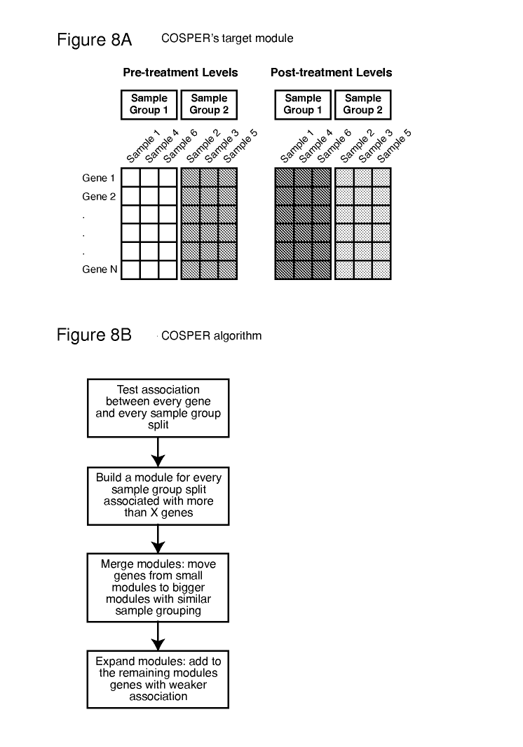

Using The Cancer Genome Atlas (TCGA) melanoma dataset, we associated STAT1 pathway activity levels with genetic aberrations. To infer pathway activity, we used the genes in the STAT1 cluster identified by COSPER, which reflect pSTAT1 levels are also highly correlated in the TCGA patient derived dataset (FIG. 7A). With a substantially increased number of samples, this patient-derived dataset enables a genome-wide search for loci whose copy number levels are associated with STAT1 activity (see experimental procedures).

The copy number aberration most significantly associated with the STAT1 gene signature is a deletion of the interferon locus (q-value=10⁻⁴, FDR (Storey and Tibshirani, 2003)), located in chromosome 9p22. The locus contains a cluster of 26 interferon genes (FIG. 7B) and deletion of this locus corresponds to low basal activity of the interferon pathway. To validate the association, we assessed copy number levels of our cell line panel using CGH arrays. Our panel confirms this association—most cell lines with low pathway activity have 0 or 1 copies of the 9p22 locus, while all cell lines with high activity have 2 or 3 copies (FIG. 7C, see experimental procedures for copy number assessment).

Interestingly, the interferon gene cluster on locus 9p22 is only 0.5 Mbs downstream of p16 (CDKN2A) (FIG. 7B), a known tumor suppressor gene deleted in roughly 60% of melanoma tumors (Reed et al., 1995). Deletion of both p16 and the interferon locus was previously reported (Naylor et al., 1997), but as research focused on the role p16 in cancer, deletion of the interferon locus was viewed as a passenger mutation. However, copy number data show that both events are independent, and copy number of the interferon locus and not p16 is associated with the phenotypic response to MEK inhibition (FIG. 7C).

Deletion of the interferon locus leads to lower expression levels of the interferon genes (FIG. 14B), which can explain the low pSTAT1 levels in those cell lines. To confirm that an autocrine loop is responsible for the lower levels of pSTAT1, we performed a conditioned media experiment. In these experiments media from high pSTAT1 cell lines lead to activation of STAT1 in low pSTAT1 cell lines (FIG. 14C), confirming that high pSTAT1 cell lines that harbor two copies of the interferon locus produce and release cytokines, presumably IFN, which leads to STAT1 activation.

To summarize, our results show that cell lines with fewer copies of the interferon locus and without expression of the interferon genes are sensitive to the cytotoxic effects of MEK inhibition (FIG. 7D). Furthermore, IFNα/β enhances this cytotoxic response via an increase in CytoC release from the mitochondria. However, cell lines with high basal activity of the interferon pathway are resistant to the cytotoxic effects of the treatments, and although MEK inhibition leads to CytoC release in these cell lines, it seems that an impairment of the caspase activation mechanism leads to apoptosis aversion. Taken together, we postulate that constitutive exposure to IFN is adverse to cancer cells, and they overcome it by either deactivation of the interferon pathway, or by an impairment of the apoptotic pathway.

### DISCUSSION

Contemporary cancer drug development focuses on targeting recurring oncogenic events, such as gene amplification and overexpression (HER2) or activation (BRAF). This approach is based on the principle of oncogene addiction. The underlying assumption is that, both the network structure and the downstream targets of the oncogenes, are the same in all tumors. Taken further, drug combinations are also currently suggested based on the principle of similar network structure and pathway dependencies in tumors harboring a specific oncogenic mutation.

However, our analysis of MAPK targets in MAPK-activated melanomas reveals tremendous differences in underlying network structure between tumors. Although we analyzed the transcriptional output of MEK inhibition only in melanoma cell lines with MAPK activating mutations (BRAF or NRAS), each cell line had a unique transcriptional signature. Moreover, a vast majority of downstream targets of the MAPK pathway are context-specific—under the control of the pathway in only a subset of cell lines. We showed that these differences could help explain the phenotypic heterogeneity observed in vitro.

To detect context-specific targets using pre- and post-inhibition expression data, we developed COSPER, a bi-clustering algorithm that identifies co-expressed genes that are under the control of the MAPK pathway in only a subset of cell lines. There are four benefits to identifying clusters of context-specific, co-regulated genes. First, we can apply enrichment analysis to the co-expressed genes and identify the cellular process or pathway that likely regulates their expression. Second, by using post-inhibition data to narrow the gene set to only those that respond to perturbation, we specifically search for pathways and processes regulated by the MAPK pathway. Third, the context—partitioning the cell lines into two groups, can assist in the identification of genetic aberrations that are more frequent in one group versus the other, thus also associating a genetic lesion with pathway activation. Fourth, the subgrouping of cell lines can also be associated with a phenotype, such as growth rate, response to treatment, “stem cell-ness” and others. Together, context-specific co-regulated clusters link genetic lesions to a MAPK-regulated pathway and a phenotype, and can assist in the understanding of response heterogeneity.

Using COSPER, we identified a possible interaction between MEK inhibition and IFN treatment, two approved treatments for melanoma. An experimental validation uncovered two key findings: first, IFNα/β enhances the cytotoxic response of MEK inhibition; second, cell lines with high basal activity of the interferon pathway exhibit much lower cytotoxicity under MEK inhibition. We found that a deletion of the interferon locus is correlated, and explains, the basal activity level of the interferon pathway, and therefore predicts the cytotoxic response to MEK inhibition. However, our results indicate that the basal activity level is not the mechanism for the sensitivity and resistance to IFNα/β and MEK inhibition. Instead, we found an impairment of the caspase activation mechanism that may explain the cytotoxic resistance.

We found that MEK inhibition leads to, and IFNβ increases, the release of CytoC from the mitochondria in all cell lines, regardless of their interferon-pathway basal activity level. Following CytoC release, caspases 9, 7 and 3 are activated only in cell lines with low interferon pathway activity. Cell lines with high basal pathway activity, however, do not cleave and activate the caspase cascade following MEK inhibition, and apoptosis is averted. We failed, however, to identify the lesion that prevents caspase activation. Understanding the mechanism of resistance can support the development of new drugs and treatments.

Taken together, these results suggest that constitutive exposure to IFN is adverse to cancer cells, and they overcome it by either deactivation of the interferon pathway, or by an impairment of the apoptotic pathway. Interferon pathway activity was previously linked to drug response. Weichselbaum et al. (Weichselbaum et al., 2008) found that interferon pathway activity predicts survival of breast cancer patients following chemotherapy and radiation. Our analysis of the TCGA data show that a lower basal activity of the interferon pathway in breast cancer is associated with a deletion of IRF1, Interferon Response Factor 1, a necessary protein for interferon-induced death (data not shown) (Sanceau et al., 2000).

The interferon pathway may have important clinical implications in melanoma and other cancers. Since interferon pathway activity predicts the cytotoxic response to MEK inhibition in vitro, it is possible that its signaling activity, interferon expression levels and/or interferon locus copy number can be used as a biomarker for treatment by MAPK pathway inhibitors.

To summarize, our work demonstrates that tumor networks are more complex and varied than previously appreciated, even within a subtype of cancer that shares key oncogenic mutations. Although only MAPK-activated melanoma cell lines were examined, these were found to be heterogeneous and immensely varied. Moreover, while all BRAF-mutant tumors are grouped together and treated similarly in the clinic, the targets and pathways regulated by BRAF in different cell lines are vastly different. Even with a small sample size of only 14 cell lines, pre- and post-perturbation expression data empowers the discovery of dependencies and interactions between pathways.

Post-perturbation data significantly enhance the ability to identify downstream targets (Niepel et al., 2013; Sachs et al., 2005). Perturbations break correlated patterns, resolve cause and effect, and reveal regulation patterns that are not observed in steady state expression levels. It was previously shown that response to perturbation varies significantly, even in cancer subtypes that share similar oncogenic mutations (Duncan et al., 2012; Niepel et al., 2014). However, analysis of post perturbation protein levels typically focus only on post-perturbation changes, regardless of pathway activation prior to perturbation., When an important pathway such as MAPK is inhibited, many of the differentially expressed genes involve response to stress, rather than genes that were regulated by the pathway prior to the perturbation. Typical methods would consider these MAPK targets (and indeed these respond to MAPK inhibition), however these are not regulated by MAPK in physiological conditions, prior to MAPK inhibition. COSPER can distinguish these using expression patterns prior to perturbation. Moreover, COSPER takes context into account. This allows us to identify clusters that only change in subsets of cell lines that would likely be dismissed by other methods. By comparing both the pre- and post-perturbation gene expression, and taking context into account, we can better identify pathways that are regulated by MAPK in each cancer cell line. Therefore, by combining information from both pre- and post-perturbation levels we reveal the network structure governed by MAPK, and the differences in this structure in difference cell lines.

The full scale of these differences is only revealed when examining a perturbed network, which highlights the importance of post-inhibition data, compared with steady-state data only. Our data demonstrate the value of system-wide perturbation analysis of tumors in the era of personalized medicine.

### Materials and Methods

### Cell Culture and Drug Treatment

All cell lines were maintained in RPMI 1640 (Invitrogen 21870-092), supplemented with 2 mM glutamine, 50 units/mL penicillin, 50 units/mL streptomycin, and 10% FBS (Omega Scientific), and incubated at 37° C. in 5% CO2.

Samples for protein and gene expression analysis were plated at 60-80% confluency and incubated for 20-24 h.

For drug treatments, the concentrations were: PD325901 (50 nM), Interferon alpha (20000 U/mL, R&D 11100), Interferon beta (1000 U/mL, R&D 11415), and PLX4720 (2 μM). Control samples were collected untreated at time of treatment.

### Gene Expression and Microarrays

Samples for microarrays were harvested 8 h post treatment. RNA was extracted using a Qiagen RNeasy kit, and labeled using Agilent's one-color labeling protocol. Labeled cRNA was hybridized to Agilent's 8×60 human gene expression arrays. MEK inhibition and basal state expression levels were measured in biological duplicates. Data normalization is described in supplementary material. Genatomy was used for data visualization and enrichment analysis (Litvin et al., 2009).

We used Agilent's 1M SurePrint CGH arrays to assess copy number. DNA was extracted using Qiagen's DNeasy kit and labeled and hybridized according to Agilent's protocol. All microarray data are available on GEO under accession number GSE51115.

### TCGA Data Analysis

TCGA expression and CGH data were downloaded from the TCGA website. Genes for the STAT1 gene signature were a subset of COSPER's STAT1 signature. All genes with a Pearson r²>0.5 with at least 3 additional genes were included. Association with copy number was performed using Pearson correlation between the mean of the gene signature and copy number levels of each gene. Pearson's p-values were corrected by FDR (Storey and Tibshirani, 2003).

### Protein Levels

Samples for protein analysis were lysed using RIPA buffer. Protein concentration was assessed using BCA staining Samples were then normalized to a fixed concentration and mixed with a 5× glycerol/SDS/DTT loading buffer. Lysates were run on gradient (4-12%) Bis-Tris gels. Primary antibodies are listed in Table S2. After incubation with horseradish peroxidase-conjugated secondary antibodies, proteins were detected using chemiluminescence.

TABLE S2
list of antibodies

| Antibody | Company | Catalog number |
|---|---|---|
| Casp 7 (cleaved) | cell signaling | 9492 |
| Casp 9 (cleaved) | cell signaling | 7237 |
| Cytochrome C | abcam | ab110325 |
| GAPDH | cell signaling | 5174 |
| IRF1 | cell signaling | 8478 |
| MITF | abcam | ab12039 |
| pSTAT1 | cell signaling | 9167 |
| pSTAT3 Y705 | cell signaling | 9138 |
| STAT1 | cell signaling | 9175 |
| STAT3 | cell signaling | 9139 |

Cytochrome C release was assessed on fresh unfrozen pellets using Sucrose/Mannitol buffer (Majewski et al., 2004). Full details in the supplementary material.

### Growth Curves and Apoptosis Levels

For growth curve measurement, 50K cells were plated in 6-well plates with 2 mL of growth media. Cells were counted every 24 h following treatment using a cell counter (Coulter Z1), in triplicates.

Apoptosis was assessed by TUNEL staining Cells were plated in 6-well plates at 200K cells/well. 24 h after plating cells were treated with PD325901, and both floating and adherent cells were collected 72 h after treatment. TUNEL was performed using Invitrogen BrdU TUNEL kit.

### Microarray Preprocessing

Agilent one-color human mRNA expression 8×60 arrays were used to assess expression levels. Biological duplicates of control and MEK inhibition (MEKi) samples were used (expect for Colo829 and SkMel28 that were added to the panel after the first batch). Samples for the IFNβ microarrays were collected 8 h after treatment (with IFNβ, PD325901 or both), and a single sample was used for each.

Agilent's software was used to assess raw signal intensity. Preprocessing of both the MEKi panel and the IFN experiment was similar. Each of the 3 batches were processed independently—MEKi panel 1, MEKi panel 2 and the IFN panel.

Preprocessing consists of 3 steps—probe filtering, data normalization and probe averaging.

### Probe Filtering

Log 2 values were used from this point on. Probes were filtered based on their values. Probes with low or high levels in more than 20% of samples were removed. This was done to remove noisy and saturated probes. The lower and upper thresholds were different in different batches, depending on labeling, hybridization and scan levels:

| Batch | Lower threshold | Upper threshold |
|---|---|---|
| MEKi panel 1 | 6 | 16 |
| MEKi panel 2 | 7 | 18 |
| IFN panel | 7 | 17.5 |

Additionally, the Agilent probe flags were used to filter probes by a similar method: probes flagged in more than 20% of samples were removed. Flags that were used: will_above_bg, is_saturated, is_feat_non_uniform, is_feat_popn.

A “rescue” step was used to return probes representing genes that no probe was left to represent them. Probes representing the same gene with a high correlation (Pearson>0.75) were rescued. Additionally, probes with high SD (>3) were also rescued.

### Data Normalization

The 75th percentile of all samples was set to the average 75% by multiplying the values by a constant.

### Probe Averaging

Probes that measure the level of the same gene were averaged or filtered out. If the average Pearson correlation between all probes is >0.75, probes are averaged. If it is lower, the probe with the lowest correlation is removed. Process repeats till probes are averaged or only two probes are left. If only two probes left and the correlation is low, the probe with the higher raw intensity is chosen.

### Merging Duplicates

Baseline expression levels are mean-normalized at the gene level. Fold change is calculated against the control (baseline expression) of the cell line. Data from the two MEKi panels are combined at this point by averaging the baseline expression and fold change data.

### COSPER—Context-Specific Regulation

In COSPER, all genes are scored for all possible splits using both pre- and post-treatment expression using the NormalGamma function. Genes with a strong association with a split joins its cluster. Then, similar clusters are merged, leaving fewer clusters with more genes each.

COSPER—COntext SPEcific Regulation—is designed to identify genes that are directly regulated by the MAPK pathway (or any other perturbed pathway) in only a subset of cell lines. It is based on the assumption that genes under the direct control of a pathway are correlated before pathway inhibition and show a correlated expression change after pathway inhibition. Since we are looking for genes under the control of the pathway in only a subset of cell lines, we expect expression changes in only these cell lines.

COSPER uses pre-perturbation data to limit the search for genes under direct regulation of the perturbed pathway. After inhibition of a key signaling pathway such as MAPK, cellular events, such as metabolism, cell cycle and apoptosis, lead to expression changes of thousands of genes. Although the expression of those genes changes after MAPK inhibition, they are not directly regulated by MAPK. However, genes under the direct control of MAPK pathway depend on its activation levels both before and after inhibition of the pathway. For example, HEY1 (FIG. 4A) is under the control of MAPK in only a subset of cell lines. In HEY1 case, it is overexpressed by MAPK in cell lines with high MITF levels. Therefore, only in MITF-high cell lines, HEY1 expression levels decrease after MEK inhibition. Both pre- and post-inhibition expression levels are needed in order to determine this relationship.

COSPER is therefore designed to find genes with context-specific regulation patterns (FIG. 3B). It is consists of 3 major steps:
1. Gene level—identify binary splits with high scores for both baseline expression and fold change and construct clusters.
2. Merge related clusters—allows removal of spurious correlations and averaging the noise caused due to the small sample size.
3. Add high scoring genes to the remaining clusters
   A detailed description of each of the steps follows the section on the NormalGamma score.

### NormalGamma Score

The algorithm is based on the NormalGamma score (DeGroot, 2004; Segal et al., 2003). The NormalGamma is a Bayesian score that takes variance, mean and number of data points into account. It gives a higher score to a data matrix with low variance.

We use this score since we are looking to reduce the variance of the samples. Our algorithm searches for genes that behave similarly in a subset of samples. For example, we are looking for a subset of samples where a predefined set of genes is up-regulated, compared with the rest of the samples where the genes are not under pathway control. Mathematically, this problem can be viewed as a subset of samples where the data have a lower variance compared with the variance of all samples combined. The NormalGamma score is driven mainly by data variance and is thus suitable for our needs.

The score:

$$N = size ( data )$$

$$β = max ( 1 , \frac{λ ( α - 2 )}{λ + 1} )$$

$${β}_{plus} = β + \frac{Var ( data ) N}{2} + \frac{N {λ [ data ]}^{2}}{2 ( N + λ )}$$

$${α}_{plus} = α + \frac{N}{2}$$

$$NormalGamma ( data , λ , α ) = - N * ln ( \sqrt{2 π} ) + \frac{ln ( \frac{λ}{λ + N} )}{2} + ln ( Γ ( {α}_{plus} ) ) - ln ( Γ ( α ) ) + α ln ( β ) - {α}_{plus} ln ( {β}_{plus} )$$

The score used to assess the quality of the split is:

NormalGamma (right samples)+NormalGamma (left samples)−NormalGamma(all samples)

### Step 1: Creating Clusters

First, gene expression is normalized. Basal expression levels of each gene are set to have μ=0 and σ²=1. Fold change for each gene is standardized only (σ²=1).

Next, clusters are built bottom-up—genes are assigned to “splits”, and a split with more than one gene assigned to it is considered a cluster. However, in order to filter out spurious associations we only consider clusters with 5 or more genes. All genes are tested across all valid binary splits. A valid split assigns at least 2 samples to each sample group. The test is based on permutations and the NormalGamma score.

A gene is assigned to a split if its NormalGamma scores (as defined in the previous section) in both the baseline expression and fold change are better than 99% of the split permutations (pvalue<0.01). Additionally, in order to keep the best split-gene pairs only, an additional threshold is used:

NormalGamma (right)+NormalGamma (left)−NormalGamma(all samples)>0

To determine whether clusters with more than 5 genes can be constructed by chance. We permuted the samples in the fold change expression data and performed this step on the permutated data. No clusters with 5 or more genes were constructed. Hence we believe the resulting clusters represent biological phenomenon.

### Step 2: Merging Clusters

A gene assigned to a split is very likely to be assigned to similar splits. A similar split might have one or more samples switching “sides” (FIG. 4A). Each split has 13 similar splits with a distance=1, where one sample has switched sides, and 91 splits with distance=2.

The NormalGamma score is not strong enough to discriminate between the “true” split and neighboring splits, since the distribution of scores is very tight. However, we can assume that a gene is more likely to be assigned to the real biological split, and less likely to be associated with a split with a distance>0 from the real split. We also work under the assumption that a true biological “context” is likely to influence many genes, and therefore larger clusters are more biologically relevant.

We use these two assumptions in order to identify the real gene-split associations and remove irrelevant clusters.

The cluster merging algorithm is an iterative process. Each cycle identifies the largest cluster, its genes are removed from all its neighboring clusters, and the process iterates till no more clusters can be identified.

The steps are:
1. Each cluster is scored based on its overlap with its neighbors:

$$Score ( {cluster}_{x} ) = {\sum }_{i where Distance ( {Split}_{X} , {Split}_{i} ) \leq d} {( { }_{{Cluster}_{x}} ⋂ {Genes}_{{Cluster}_{i}} )}^{}$$

we used d=2.
2. We then choose the largest cluster, and remove its genes from all clusters with a distance<=d.

To save computing time, only clusters that enter the algorithm with 5 or more genes are allowed to be selected.

### Step 3: Adding Genes to Remaining Clusters

In the last step, after filtering most clusters out, we allow genes from neighboring clusters to be added back to clusters. We found this step to be necessary due to the small sample size, the overall small distance between clusters, the relatively high noise of gene expression data, and the inability of the NormalGamma score to discriminate between similar splits.

Genes belonging to clusters in a distance<=d of a specific cluster, and with a p-value<0.01 are added to this cluster.

### Perturbation Data Allows Better Cluster Identification

Combining pre- and post-inhibition data facilitates the identification of context-specific regulation and differential activation of pathways, while pre-inhibition data alone fall short due to lower specificity and much higher rate of false positive.

For example, when running the first step of COSPER on pre-inhibition data alone, the STAT3 cluster contains 766 genes, compared with 28 genes when using both datasets. While the smaller cluster is enriched for STAT3-related terms, the larger pre-inhibition-data cluster is enriched for general terms such as “extracellular region” and “plasma membrane”.

The combination of pre- and post-inhibition data, therefore, provides specificity and limits the cluster genes to only genes directly regulated by MAPK, while also provides the context of regulation.

Comparison of BRAF and MEK Inhibition—PLX4720 vs. PD325901

We used PD325901 to inhibit the MAPK pathway, and not the more clinically used PLX4720 BRAF-V600E inhibitor to allow a direct comparison of BRAF and NRAS mutant cell lines. To ensure the short-term drug effects are similar, we compared the transcriptional response of Ma1Me3M, a BRAF-V600E cell line, following PD325901 or PLX4720 treatment. We assessed expression fold change at 1 hour, 2, 4, and 8 hours following treatment using Illumina HumanHT-12 microarrays.

### Preprocessing

Illumina's probe pvalues were used to filter out probes. Probes with p-value>0.05 in more than half of the samples were removed. Then microarrays were normalized according to their 75% percentile values. The 2 control array were averaged, and treated samples were compared to the averaged control to assess fold change.

### Results

MEKi and BRAFi are remarkably the same at all time points. Although some probes were noisy, resulting in minor difference between treatments, no gene had a difference greater than 0.5 fold (on a log 2 scale) between treatments at all time points. Only 6 probes, out of 16000, had a difference of more than 1 fold at 8 hour time point (FIG. 9B). None of them had such difference at 4 hours, suggesting that the difference arises from measurement noise.

We conclude that there is no difference in the short-time transcriptional response between treatments in this cell lines.

### Comparison of the Response to MEK Inhibition Between Known Genetic Contexts

Both inactivation of PTEN and the type of MAPK activation (BRAF or NRAS) have been previously associated with the response to MAPK pathway inhibition. We examined whether these mutations are correlated with the transcriptional response to MEK inhibition or the basal expression levels prior to MEK inhibition.

We used t-test to compare the expression levels between BRAF- and NRAS mutant cell lines (FIG. 10A), and between PTEN-null and PTEN-wild type cell lines (FIG. 10B). In both cases we found that no genes are associated with those genetic contexts (FDR q-value <0.05), either before or after pathway inhibition.

### PD325901 and IFNβ Microarray Results

### Data Preprocessing

Six cell lines were chosen for analysis. 3 are low-pSTAT1—A375, SkMel33 and SkMel2, and 3 high-pSTAT1—SkMel105, SkMel39 and WM1361. They were treated with 50 nM PD325901, 1000U/mL IFNβ or their combination. Samples were collected 8 hours after treatment, control samples were collected at 0 h. RNA extraction, labeling and hybridization were conducted as described under methods. Agilent human 8×60 gene expression arrays were used. Raw data normalization and filtering were conducted as described above, with a low threshold of 7, and an upper threshold of 17.5.

IFN Response in High- Vs. Low-pSTAT1 Cell Lines

The IFN response includes dozens of genes with a dramatic induction in gene expression, of up to 500 fold, in all 6 cell lines (FIG. 13B).

There is, however, a difference in the extent of change in high- vs. low-pSTAT1 cell lines that can be attributed to the different basal expression level of those genes. The maximum level of expression seems to be similar in all cell lines, but high pSTAT1 cell lines have a higher basal activity and therefore the fold change is lower.

In order to compare the activation of the pathway between the two cell line groups, it is better to use the final expression level, i.e. the basal expression+fold change. However, such comparison reveals the expression of no genes is statistically significant different between high- and low-pSTAT1 cell lines (using t-test and FDR correction).

We therefore determine that there is no difference in the response to IFNβ between high- and low-pSTAT1 cell lines.

### Combinatorial Treatment and Synergy

To test whether the MEK inhibition and IFNβ synergize at the level of gene expression, we compared the fold change of the dual treatment with that of MEKi and IFNβ as single agents. Over all, those responses are very similar (FIG. 13C).

If no synergy exists, the values of gene expression fold change after treatment by Both Agents—(gene expression fold change after MEKi treatment alone+gene expression fold change after IFNβ treatment alone) should be close to 0. Only one gene significantly deviates from 0 in all 6 cell lines. The gene is CCL4, and it is induced both by MEKi and IFNβ treatment as single agents, but a combinatorial treatment is not additive.

We could not identify any other genes that show a synergetic response in all 6 cell lines, or separately in low- or high-pSTAT1 lines.

### MITF Binding Site Analysis

To assess frequency of MITF binding site in gene promoters we used the motif CACATG, known to be a target sequence of MITF. Gene promoters were defined as 5000 bp upstream of their transcription start site, or up to the closest upstream gene, whichever is shorter. For each gene, number of binding motif in its promoter sequence was noted.

To assess the significance of number of motif occurrences, we used the binomial distribution. For each one of the two clusters, MITF-M and MITF-expression, we counted total number of motif occurrences in all the cluster genes. For simplicity, the expected probability of the motif to randomly appear in a DNA sequence is 2*1/4⁶ (6 is the length of the motif, and 2 represent the two strands).

The p-value of X occurrences is the probability of randomly observing X or more occurrences in a random sequence, or 1-BINOMIAL_CDF(X, N, p), where N is total sequence length and p is 2/4⁶.

For MITF-M cluster, the total promoter sequence is 120735 bp, with 83 motif occurrences (59 expected). For MITF-expression cluster, the total promoter sequence is 183399 bp, with 86 occurrences (89 expected).

### Cytochrome C Release

Protocol for Cytochrome C release is taken, as is, from Majewski et al 2004. Lysis buffer: 20 mM Hepes-KOH, [pH 7.5], 210 mM sucrose, and 70 mM mannitol; 1.5 mM MgCl2, 10 mM KCl2, protease inhibitor, and 1 mg digitonin/1 mL lysis buffer.

Cells are trypsinized, collected and spun down in 4C. They are then washed with PBS and spun down again. It is critical that cell pellets will be lysed immediately without freezing. Cells are gently suspended, without vortexing, in lysis buffer. Roughly double the cell pellet volume is used. They are incubated in 25C for 3-10 min, depending all cell line. Spun down at 4C for 20 minutes at highest speed. Supernatant contains cytoplasmic fraction.

Protein concentration was assessed using BCA.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

### REFERENCES

Barretina, J., Caponigro, G., Stransky, N., Venkatesan, K., Margolin, A. A., Kim, S., Wilson, C. J., Lehar, J., Kryukov, G. V., Sonkin, D., et al. (2012). The Cancer Cell Line Encyclopedia enables predictive modelling of anticancer drug sensitivity. Nature 483, 603-607.
Bratton, S. B., and Salvesen, G. S. (2010). Regulation of the Apaf-1-caspase-9 apoptosome. Journal of cell science 123, 3209-3214.
Corcoran, R. B., Ebi, H., Turke, A. B., Coffee, E. M., Nishino, M., Cogdill, A. P., Brown, R. D., Della Pelle, P., Dias-Santagata, D., Hung, K. E., et al. (2012). EGFR-mediated re-activation of MAPK signaling contributes to insensitivity of BRAF mutant colorectal cancers to RAF inhibition with vemurafenib. Cancer discovery 2, 227-235.
Dai, B., Meng, J., Peyton, M., Girard, L., Bornmann, W. G., Ji, L., Minna, J. D., Fang, B., and Roth, J. A. (2011). STAT3 mediates resistance to MEK inhibitor through microRNA miR-17. Cancer research 71, 3658-3668.
Duncan, J. S., Whittle, M. C., Nakamura, K., Abell, A. N., Midland, A. A., Zawistowski, J. S., Johnson, N. L., Granger, D. A., Jordan, N. V., Darr, D. B., et al. (2012). Dynamic reprogramming of the kinome in response to targeted MEK inhibition in triple-negative breast cancer. Cell 149, 307-321.
Flaherty, K. T., Puzanov, I., Kim, K. B., Ribas, A., McArthur, G. A., Sosman, J. A., O'Dwyer, P. J., Lee, R. J., Grippo, J. F., Nolop, K., et al. (2010). Inhibition of mutated, activated BRAF in metastatic melanoma. The New England journal of medicine 363, 809-819.
Garnett, M. J., Edelman, E. J., Heidorn, S. J., Greenman, C. D., Dastur, A., Lau, K. W., Greninger, P., Thompson, I. R., Luo, X., Soares, J., et al. (2012). Systematic identification of genomic markers of drug sensitivity in cancer cells. Nature 483, 570-575.
Hecker, M., Hartmann, C., Kandulski, O., Paap, B. K., Koczan, D., Thiesen, H. J., and Zettl, U. K. (2013). Interferon-beta therapy in multiple sclerosis: the short-term and long-term effects on the patients' individual gene expression in peripheral blood. Molecular neurobiology 48, 737-756.
Hodis, E., Watson, I. R., Kryukov, G. V., Arold, S. T., Imielinski, M., Theurillat, J. P., Nickerson, E., Auclair, D., Li, L., Place, C., et al. (2012). A landscape of driver mutations in melanoma. Cell 150, 251-263.
Jackson, D. P., Watling, D., Rogers, N. C., Banks, R. E., Kerr, I. M., Selby, P. J., and Patel, P. M. (2003). The JAK/STAT pathway is not sufficient to sustain the antiproliferative response in an interferon-resistant human melanoma cell line. Melanoma research 13, 219-229.
Johannessen, C. M., Johnson, L. A., Piccioni, F., Townes, A., Frederick, D. T., Donahue, M. K., Narayan, R., Flaherty, K. T., Wargo, J. A., Root, D. E., et al. (2013). A melanocyte lineage program confers resistance to MAP kinase pathway inhibition. Nature 504, 138-142.
Joseph, E. W., Pratilas, C. A., Poulikakos, P. I., Tadi, M., Wang, W., Taylor, B. S., Halilovic, E., Persaud, Y., Xing, F., Viale, A., et al. (2010). The RAF inhibitor PLX4032 inhibits ERK signaling and tumor cell proliferation in a V600E BRAF-selective manner. Proceedings of the National Academy of Sciences of the United States of America 107, 14903-14908.
Leaman, D. W., Chawla-Sarkar, M., Jacobs, B., Vyas, K., Sun, Y., Ozdemir, A., Yi, T., Williams, B. R., and Borden, E. C. (2003). Novel growth and death related interferon-stimulated genes (ISGs) in melanoma: greater potency of IFN-beta compared with IFN-alpha2. Journal of interferon & cytokine research: the official journal of the International Society for Interferon and Cytokine Research 23, 745-756.
Levy, C., Khaled, M., and Fisher, D. E. (2006). MITF: master regulator of melanocyte development and melanoma oncogene. Trends in molecular medicine 12, 406-414.
Lito, P., Pratilas, C. A., Joseph, E. W., Tadi, M., Halilovic, E., Zubrowski, M., Huang, A., Wong, W. L., Callahan, M. K., Merghoub, T., et al. (2012). Relief of profound feedback inhibition of mitogenic signaling by RAF inhibitors attenuates their activity in BRAFV600E melanomas. Cancer cell 22, 668-682.
Litvin, O., Causton, H. C., Chen, B. J., and Pe'er, D. (2009). Modularity and interactions in the genetics of gene expression. Proceedings of the National Academy of Sciences of the United States of America 106, 6441-6446.
Majewski, N., Nogueira, V., Bhaskar, P., Coy, P. E., Skeen, J. E., Gottlob, K., Chandel, N. S., Thompson, C. B., Robey, R. B., and Hay, N. (2004). Hexokinase-mitochondria interaction mediated by Akt is required to inhibit apoptosis in the presence or absence of Bax and Bak. Molecular cell 16, 819-830.
Naylor, M. F., Brown, S., Quinlan, C., Pitha, J. V., and Evertt, M. A. (1997). 9p21 deletions in primary melanoma. Dermatology online journal 3, 1.
Niepel, M., Hafner, M., Pace, E. A., Chung, M., Chai, D. H., Zhou, L., Muhlich, J. L., Schoeberl, B., and Sorger, P. K. (2014). Analysis of growth factor signaling in genetically diverse breast cancer lines. BMC biology 12, 20.
Niepel, M., Hafner, M., Pace, E. A., Chung, M., Chai, D. H., Zhou, L., Schoeberl, B., and Sorger, P. K. (2013). Profiles of Basal and stimulated receptor signaling networks predict drug response in breast cancer lines. Science signaling 6, ra84.
Paraiso, K. H., Xiang, Y., Rebecca, V. W., Abel, E. V., Chen, Y. A., Munko, A. C., Wood, E., Fedorenko, I. V., Sondak, V. K., Anderson, A. R., et al. (2011). PTEN loss confers BRAF inhibitor resistance to melanoma cells through the suppression of BIM expression. Cancer research 71, 2750-2760.
Platanias, L. C. (2005). Mechanisms of type-I- and type-II-interferon-mediated signalling. Nature reviews Immunology 5, 375-386.
Poulikakos, P. I., Zhang, C., Bollag, G., Shokat, K. M., and Rosen, N. (2010). RAF inhibitors transactivate RAF dimers and ERK signalling in cells with wild-type BRAF. Nature 464, 427-430.
Pratilas, C. A., Taylor, B. S., Ye, Q., Viale, A., Sander, C., Solit, D. B., and Rosen, N. (2009). (V600E) BRAF is associated with disabled feedback inhibition of RAF-MEK signaling and elevated transcriptional output of the pathway. Proceedings of the National Academy of Sciences of the United States of America 106, 4519-4524.
Reed, J. A., Loganzo, F., Jr., Shea, C. R., Walker, G. J., Flores, J. F., Glendening, J. M., Bogdany, J. K., Shiel, M. J., Haluska, F. G., Fountain, J. W., et al. (1995). Loss of expression of the p16/cyclin-dependent kinase inhibitor 2 tumor suppressor gene in melanocytic lesions correlates with invasive stage of tumor progression. Cancer research 55, 2713-2718.
Riedl, S. J., and Shi, Y. (2004). Molecular mechanisms of caspase regulation during apoptosis. Nature reviews Molecular cell biology 5, 897-907.
Sachs, K., Perez, O., Pe'er, D., Lauffenburger, D. A., and Nolan, G. P. (2005). Causal protein-signaling networks derived from multiparameter single-cell data. Science 308, 523-529.
Sanceau, J., Hiscott, J., Delattre, O., and Wietzerbin, J. (2000). IFN-beta induces serine phosphorylation of Stat-1 in Ewing's sarcoma cells and mediates apoptosis via induction of IRF-1 and activation of caspase-7. Oncogene 19, 3372-3383.
Slamon, D. J., Leyland-Jones, B., Shak, S., Fuchs, H., Paton, V., Bajamonde, A., Fleming, T., Eiermann, W., Wolter, J., Pegram, M., et al. (2001). Use of chemotherapy plus a monoclonal antibody against HER2 for metastatic breast cancer that overexpresses HER2. The New England journal of medicine 344, 783-792.
Soengas, M. S., Capodieci, P., Polsky, D., Mora, J., Esteller, M., Opitz-Araya, X., McCombie, R., Herman, J. G., Gerald, W. L., Lazebnik, Y. A., et al. (2001). Inactivation of the apoptosis effector Apaf-1 in malignant melanoma. Nature 409, 207-211.
Solit, D. B., Garraway, L. A., Pratilas, C. A., Sawai, A., Getz, G., Basso, A., Ye, Q., Lobo, J. M., She, Y., Osman, I., et al. (2006). BRAF mutation predicts sensitivity to MEK inhibition. Nature 439, 358-362.
Sosman, J. A., Kim, K. B., Schuchter, L., Gonzalez, R., Pavlick, A. C., Weber, J. S., McArthur, G. A., Hutson, T. E., Moschos, S. J., Flaherty, K. T., et al. (2012). Survival in BRAF V600-mutant advanced melanoma treated with vemurafenib. The New England journal of medicine 366, 707-714.
Storey, J. D., and Tibshirani, R. (2003). Statistical significance for genomewide studies. Proceedings of the National Academy of Sciences of the United States of America 100, 9440-9445.
Tsai, J., Lee, J. T., Wang, W., Zhang, J., Cho, H., Mamo, S., Bremer, R., Gillette, S., Kong, J., Haass, N. K., et al. (2008). Discovery of a selective inhibitor of oncogenic B-Raf kinase with potent antimelanoma activity. Proceedings of the National Academy of Sciences of the United States of America 105, 3041-3046.
Weichselbaum, R. R., Ishwaran, H., Yoon, T., Nuyten, D. S., Baker, S. W., Khodarev, N., Su, A. W., Shaikh, A. Y., Roach, P., Kreike, B., et al. (2008). An interferon-related gene signature for DNA damage resistance is a predictive marker for chemotherapy and radiation for breast cancer. Proceedings of the National Academy of Sciences of the United States of America 105, 18490-18495.
Xing, F., Persaud, Y., Pratilas, C. A., Taylor, B. S., Janakiraman, M., She, Q. B., Gallardo, H., Liu, C., Merghoub, T., Hefter, B., et al. (2012). Concurrent loss of the PTEN and RB1 tumor suppressors attenuates RAF dependence in melanomas harboring (V600E) BRAF. Oncogene 31, 446-457.
Zhang, J., Xiao, Z., Lai, D., Sun, J., He, C., Chu, Z., Ye, H., Chen, S., and Wang, J. (2012). miR-21, miR-17 and miR-19a induced by phosphatase of regenerating liver-3 promote the proliferation and metastasis of colon cancer. British journal of cancer 107, 352-359.
DeGroot, M. H. (2004). Optimal statistical decisions, Wiley classics library edn (Hoboken, N.J.: Wiley-Interscience).
Segal, E., Shapira, M., Regev, A., Pe'er, D., Botstein, D., Koller, D., and Friedman, N. (2003). Module networks: identifying regulatory modules and their condition-specific regulators from gene expression data. Nature genetics 34, 166-176.

What is claimed is:

1. A method of treating cancer in a subject, comprising the step of administering to the subject an interferon and an inhibitor of mitogen-activated protein kinase (MAPK) signaling pathway, wherein the combination of the interferon and the inhibitor of the MAPK pathway produces a synergistic effect on the cancer compared to the effect of the interferon alone or the effect of the inhibitor of the MAPK pathway alone.

2. The method of claim 1, wherein the combination results in a synergistic increase in apoptosis of cancer cells.

3. The method of claim 1, wherein the combination results in a synergistic reduction in tumor volume.

4. The method of claim 1, wherein the inhibitor is an inhibitor of RAF, an inhibitor of MEK, an inhibitor of ERK, an inhibitor of RAS, an inhibitor of receptor tyrosine kinases (RTKs), or combinations thereof.

5. The method of claim 1, wherein the inhibitor is a small molecule, a polynucleotide, a polypeptide, or an antibody or antigen-binding portion thereof.

6. The method of claim 1, wherein the inhibitor is PLX4720, PD325901, GW5074, BAY 43-9006, ISIS 5132, PD98059, PD184352, U0126, Ro 09-2210, L-783,277, GSK-1120212, trametinic, vemurafenib, purvalanol, or imidazolium trans-imidazoledimethyl sulfoxide-tetrachlororuthenate (NAMI-A).

7. The method of claim 5, wherein the polynucleotide is a small interfering RNA (siRNA) or an antisense molecule.

8. The method of claim 1, wherein the interferon is a type I, type II or type III interferon.

9. The method of claim 8, wherein the interferon is a type I interferon selected from the group consisting of interferon-α, interferon-β, interferon-ε, interferon-κ, and interferon-ω.

10. The method of claim 1, wherein the interferon and the inhibitor are administered simultaneously, sequentially or separately.

11. The method of claim 1, wherein the cancer is melanoma, breast cancer, colon cancer, pancreatic cancer, cervical cancer, thyroid cancer or bladder cancer.

12. A method of treating cancer in a subject, comprising the step of administering to the subject an interferon and a cytotoxic agent, wherein the combination of the interferon and the cytotoxic agent produces a synergistic effect on the cancer compared to the effect of the interferon alone or the effect of the cytotoxic agent alone.

13. The method of claim 12, wherein the cytotoxic agent is an inhibitor of MAPK signaling pathway, an alkylating agent, an anti-metabolite, an anti-microtubule agent, a topoisomerase inhibitor, a cytotoxic antibiotic, or an endoplasmic reticulum stress inducing agent.

14. The method of claim 12, wherein the combination results in a synergistic increase in apoptosis of cancer cells.

15. The method of claim 12, wherein the combination results in a synergistic reduction in tumor volume.

16. The method of claim 12, wherein the interferon is a type I interferon selected from the group consisting of interferon-α, interferon-β, interferon-ε, interferon-κ, and interferon-ω.

17. A pharmaceutical composition comprising a first amount of an interferon and a second amount of an inhibitor of the mitogen-activated protein kinase (MAPK) signaling pathway, wherein the combination of the first amount of interferon and the second amount of the inhibitor of the MAPK pathway produces a synergistic effect on cancer compared to the effect of the first amount of interferon alone or the effect of the second amount of the inhibitor of the MAPK pathway alone.

18. The pharmaceutical composition of claim 17, wherein the combination results in a synergistic increase in apoptosis of cancer cells.

19. The pharmaceutical composition of claim 17, wherein the combination results in a synergistic reduction in tumor volume.

20. The pharmaceutical composition of claim 17, wherein the inhibitor is an inhibitor of RAF, an inhibitor of MEK, an inhibitor of ERK, an inhibitor of RAS, an inhibitor of receptor tyrosine kinases (RTKs), or combinations thereof.

21. The pharmaceutical composition of claim 17, wherein the inhibitor is a small molecule, a polynucleotide, a polypeptide, or an antibody or antigen-binding portion thereof.

22. The pharmaceutical composition of claim 17, wherein the inhibitor is PLX4720, PD325901, GW5074, BAY 43-9006, ISIS 5132, PD98059, PD184352, U0126, Ro 09-2210, L-783,277, GSK-1120212, trametinic, vemurafenib, purvalanol, or imidazolium trans-imidazoledimethyl sulfoxide-tetrachlororuthenate (NAMI-A).

23. The pharmaceutical composition of claim 17, wherein the interferon is a type I interferon selected from the group consisting of interferon-α, interferon-β, interferon-ε, interferon-κ, and interferon-ω.

24. A method of treating cancer cells, comprising the steps of:
(a) determining activity of STAT1 (Signal Transduction And Transcription 1) signaling pathway in the cancer cells; and
(b) administering to the cancer cells an inhibitor of the mitogen-activated protein kinase (MAPK) signaling pathway, if the activity of the STAT1 signaling pathway in step (a) is less than 20% of activity of STAT1 signaling pathway in WM1361 melanoma cells.

25. The method of claim 24, wherein in step (b) an interferon is also administered.

26. The method of claim 24, wherein in step (a) the activity of STAT signaling pathway is determined by assaying the level of pSTAT1-Y701 (STAT1 phosphorylated at Tyr701).

27. The method of claim 24, wherein in step (a) the activity of STAT signaling pathway is determined by an assay selected from the group consisting of: (i) an assay of protein level or phosphorylation level of JAK1/2, STAT1/2 and/or interferon receptors; (ii) an assay of expression levels of STAT1/2 downstream genes; and (iii) an assay of mRNA and protein levels of interferon-α or interferon-β.

28. The method of claim 24, wherein the inhibitor is an inhibitor of RAF, an inhibitor of MEK, an inhibitor of ERK, an inhibitor of RAS, an inhibitor of receptor tyrosine kinases (RTKs), or combinations thereof.

29. The method of claim 24, wherein the inhibitor is a small molecule, a polynucleotide, a polypeptide, or an antibody or antigen-binding portion thereof.

30. The method of claim 24, wherein the inhibitor is PLX4720, PD325901, GW5074, BAY 43-9006, ISIS 5132, PD98059, PD184352, U0126, Ro 09-2210, L-783,277, GSK-1120212, trametinic, vemurafenib, purvalanol, or imidazolium trans-imidazoledimethyl sulfoxide-tetrachlororuthenate (NAMI-A).

31. The method of claim 24, wherein the interferon is a type I interferon selected from the group consisting of interferon-α, interferon-β, interferon-ε, interferon-κ, and interferon-ω.

32. The method of claim 25, wherein the interferon and the inhibitor are administered simultaneously, sequentially or separately.

33. A method of treating cancer cells, comprising the steps of:
(a) determining copy number of interferon locus located on chromosome 9p22 in the cancer cells;
(b) administering to the cancer cells an inhibitor of the mitogen-activated protein kinase (MAPK) signaling pathway, if the copy number of the interferon locus determined in step (a) is 0 or 1.

34. The method of claim 33, wherein in step (b) an interferon is also administered.

35. The method of claim 33, wherein the inhibitor is an inhibitor of RAF, an inhibitor of MEK, an inhibitor of ERK, an inhibitor of RAS, an inhibitor of receptor tyrosine kinases (RTKs), or combinations thereof.

36. The method of claim 33, wherein the inhibitor is a small molecule, a polynucleotide, a polypeptide, or an antibody or antigen-binding portion thereof.

37. The method of claim 33, wherein the inhibitor is PLX4720, PD325901, GW5074, BAY 43-9006, ISIS 5132, PD98059, PD184352, U0126, Ro 09-2210, L-783,277, GSK-1120212, trametinic, vemurafenib, purvalanol, or imidazolium trans-imidazoledimethyl sulfoxide-tetrachlororuthenate (NAMI-A).

38. The method of claim 33, wherein the interferon is a type I interferon selected from the group consisting of interferon-α, interferon-β, interferon-ε, interferon-κ, and interferon-ω.

39. The method of claim 34, wherein the interferon and the inhibitor are administered simultaneously, sequentially or separately.
